(19) United States
(12) Patent Application Publication
(10) Pub. No.: US 2026/0306279 A1
(43) Pub. Date: Oct. 8, 2026

(54) PLUG IMPLANT HOLDERS FOR REPLACING DAMAGED HYALINE CARTILAGE ON BONE SURFACES

(71) Applicant: FORMAE, INC., Paoli, PA (US)

(72) Inventor: Kevin A. MANSMANN, Paoli, PA (US)

(73) Assignee: FORMAE, INC., Paoli, PA (US)

(21) Appl. No.: 19/716,531

(22) Filed: Jun. 23, 2026

Related U.S. Application Data
(63) Continuation-in-part of application No. 19/652,387, filed on Apr. 20, 2026.
(63) Continuation-in-part of application No. 19/446,060, filed on Jan. 12, 2026.
Application No. 19/652,387 is a continuation of application No. 18/259,191, filed on Dec. 22, 2021, now Pat. No. 12,605,257.
(63) Continuation-in-part of application No. 17/781,558, filed on Dec. 7, 2020.
Application No. 19/446,060 is a continuation of application No. 15/766,066, filed on Oct. 5, 2016, now Pat. No. 12,521,248.
(60) Provisional application No. 63/130,206, filed on Dec. 23, 2020.
(60) Provisional application No. 62/944,467, filed on Dec. 6, 2019.
(60) Provisional application No. 62/237,477, filed on Oct. 5, 2015.
(60) Provisional application No. 62/237,471, filed on Oct. 5, 2015.

(51) Int. Cl. A61F 2/46; A61B 17/17; A61F 2/30

(52) U.S. Cl. CPC A61F 2/4618; A61B 17/1764; A61F 2002/30759; A61F 2002/4632; A61F 2002/4635; A61F 2002/4677

(57) ABSTRACT

An implant for use in a medical procedure is provided including an anchor body and a flexible material. The anchor body includes a gripping array of struts for anchoring a flexible material at least partially within the anchor body. The flexible material is connected to the anchor body and with at least a portion extending out of the top opening of the anchor body.

### CROSS REFERENCE TO RELATED APPLICATIONS

This application claims the benefit of priority to, and is a continuation-in-part application of, all of the following patent applications:

U.S. Non-Provisional Application Number 19/446,060, filed Jan. 12, 2026, which in turn is a continuation of and claims the benefit of priority to U.S. Non-Provisional Application Number 15/766,066, filed Apr. 5, 2018, and issued as U.S. Patent No. 12,521,248 on Jan. 13, 2026, which is in turn is a National Stage application under 35 U.S.C. § 371 and claims the benefit of priority to PCT/US2016/055403, filed Oct. 15, 2016, each of which in turn claims the benefit of priority to U.S. Provisional Application Number 62/237,471 filed Oct. 5, 2015, and also U.S Provisional Application Number, 62/237,477, filed Oct. 5, 2015; and

U.S. Non-Provisional Application Number 17/781,558, filed Jun. 1, 2022, which in turn is a National Stage application under 35 U.S.C. § 371 of and claims the benefit of priority to PCT/US2020/063539, filed Dec. 7, 2020, each of which in turn claims the benefit of priority to U.S. Provisional Application Number 62/944,467, filed Dec. 6, 2019; and

U.S. Non-Provisional Application Number 19/652,387, filed Apr. 20, 2026, which in turn is a continuation of and claims the benefit of priority to U.S. Non-Provisional Application Number 18/259,191, filed Jun. 23, 2023, and issued as U.S. Patent No. 12,605,257 on Apr. 21, 2026, which in turn is a National Stage application under 35 U.S.C. § 371 of and claims the benefit of priority to PCT/US2021/064895, filed on Dec. 22, 2021, each of which claims the benefit of priority to U.S. Provisional Application Number 63/130,206, filed Dec. 23, 2020;
the entire contents of all of which are incorporated by reference as if fully set forth herein.

### FIELD OF INVENTION

The present disclosure relates to surgical implants, and more particularly, to “plug-type” implants for replacing a specific damaged area (i.e., a lesion) of hyaline cartilage on a bone surface in an articulating joint (such as a “femoral runner” in a knee joint), without requiring an entire segment of cartilage to be replaced, as occurs in surgeries such as “total knee replacement” surgery.

### BACKGROUND

Cartilage is a flexible and non-rigid biological material, which performs a number of functions in humans and other animals. There are three main types of cartilage, and each type performs a different set of functions. “Elastic cartilage” exists mainly in the ears and windpipe, where it provides shapes and structures that need to be firm, but also flexible and resilient. “Fibro-cartilage” contains exceptionally long and thick strands of collagen (i.e., the main extra-cellular fibrous protein in animals, which holds soft tissues together); it is present in the discs positioned between the vertebral bones in a spine, and in long specialized arc-shaped segments of cartilage in knees, hips, and shoulders, called meniscal or labral segments. Those two types of cartilage are not specifically relevant to the present disclosure. The third main type of cartilage, called “hyaline cartilage” is the only type that is of interest herein.

“Hyaline cartilage” is located in thin layers which coat the surfaces of bones in “articulating” joints, such as the hips, knees, shoulders, fingers. When a limb, digit, or other body part is extended, retracted, or otherwise flexed and moved, the surfaces of two or more different bones must press against and slide across each other, in the type of motion called “articulation.” To enable smooth and painless sliding motion, the surface areas of those bones which actually engage in that sliding motion (those bone surfaces are often called “bone condyles”) are covered by thin layers of a special type of cartilage, which must remain wet and lubricated, and very smooth, to enable a joint to function properly. Since those cartilage surfaces are so smooth, slippery, and even shiny that they have an appearance similar to glass, they are called “hyaline cartilage”, from the Greek root “hyalos”, which refers to glass.

The phrase “plug implant”, as used herein, can refer to an implant device which is designed, sized, and suited for replacing only a limited area (or portion, region, spot, or similar terms; as mentioned below, a specific damaged area may also be called a lesion) of a hyaline cartilage “segment”, without having to replace that entire segment.

As used herein, a cartilage “segment” can be a continuous segment of cartilage that covers a condylar surface on some particular bone. As examples, femoral runners, and tibial plateaus, can both be regarded as cartilage “segments”; and, because of how they are shaped and structured, the “medial” half (i.e., on the inside of the knee), or the “lateral” half (on the outside of a knee), of a femoral runner, or a tibial plateau, also can be regarded and referred to as a distinct cartilage “segment.” The point that needs to be made and understood is that, because of how the phrase “plug implant” is defined and used herein, it refers to a surgical implant that is designed to be inserted into a damaged segment of hyaline cartilage, without having to replace that entire segment. As such, a plug implant must be designed to share at least one border (or edge, side, boundary, or similar terms) with an existing and undisturbed area of natural hyaline cartilage, which will not be replaced by a surgeon during that surgical operation. In many cases where a specific and limited damaged area of cartilage on a femoral runner may be replaced by a plug implant as described herein, the plug implant may be completely surrounded by unaltered native hyaline cartilage.

“Plug implants” alternately can be referred to by various other terms, if desired, such as (for example) patch implants, partial implants, button implants, bounded implants, local or localized implants, etc. For consistency, the phrase “plug implant” will be used; and, since these types of implants are the only types of surgical implants that are described and claimed herein, any reference herein to “implant” or “implant device,” when used as a noun, is limited to “plug”-type implants as described above.

A “femoral runner,” a knee joint in humans (the comparable joints in the hind legs in mammals that walk on four legs are called “stifle joints”) has several different hyaline cartilage surfaces. The femur is the long bone in the thigh, between the hip and the knee. Its lower end has two generally parallel rounded components, called the “runners” or “femoral runners” (the trough-shaped gap between them is called the trochlear groove). Both of those two “runners” slide in a tightly-constrained manner across “opposing” cartilage segments called the medial and lateral “tibial plateaus”, which sit on top of the tibia, which is the largest bone in the lower leg. The medial and lateral tibial plateaus often are collectively referred to as a single “tibial plateau”; however, they are separated by a set of raised center components (collectively called the “tibial spine”) which are not covered by cartilage. Those “tibial spine” protrusions ride inside the “trough” between the two femoral runners, and help prevent any unwanted lateral (side-to-side) motion of the femur, on top of the tibia. Additional reinforcement against unwanted lateral motion is also provided by both: (i) the ligaments of the knee; (ii) the meniscal segments, which create a flexible and generally bowl-shaped fibro-cartilage structure, which the medial and lateral sides of the femoral runners press against and slide across; and, (iii) the “patella”, commonly called the kneecap, which has rounded surfaces that the femoral runners (and the trochlear groove, between those two runners) directly press and slide against. Also, the interface between the femoral runners, and the posterior surface of the kneecap, does not have to endure compressive loads to the same extent or frequency that occurs with the tibial plateaus; however, in some postures, that interface can become a fully weight-bearing interaction.

For a number of reasons, the cartilage segments on femoral runners become damaged more than any other cartilage segments, in knee joints or any other joints. The terms “damage” and “damaged” are used broadly and inclusively herein, to cover and include any type of damage, due to any type of injury, infection, long-term wear, etc., which is serious enough to drive a sufferer to seek relief in some form that goes beyond merely “palliative” measures, such as injection of a lubricating compound or anti-inflammatory drug.

To provide additional background, orthopedic surgeons assign numerical “grades” to what they call “chondromalacia”, to indicate various levels of damage to hyaline cartilage, where “Grade 1” indicates initial softening, “Grade 2” indicates softening accompanied by signs of surface damage, and “Grade 4” indicates a major breakdown and obvious surface damage, usually causing (or threatening to soon cause) painful bone-on-bone grinding. Different people begin seeking treatment at different grade levels, in ways that often depend heavily on their insurance coverage, and financial status. Accordingly, two major points need to be recognized, to adequately understand and appreciate the invention herein:
(i) if “plug implants” can be designed and provided which are strong enough, durable enough, and sufficiently functional and pain-relieving to provide good performance, for years or even decades, even in a heavily-loaded joint such as a knee, more people suffering from knee pain or discomfort would seek help sooner; and, if good plug implants were available, people who suffer from (and do their best to endure) mild to moderate knee pain could obtain relief (and, could improve their lives, work, etc.) at a much lower cost, both to themselves and to society, rather than waiting until their knee pain is so severe that they can no longer endure it, and who then require much more expensive surgery (such as a “total knee replacement”), which requires a much longer and more painful convalescence and recovery; and,
(ii) prior efforts, before now, failed, for one reason or another (and usually for a combination of reasons, since any “plug implant” for a femoral runner will need to carefully balance out and somehow achieve multiple different and even contradictory demands and goals).

As another term that must be clarified, “lesion,” is used herein to refer to any specific area (or site, spot, or similar terms) of damaged hyaline cartilage which is targeted for replacement by a plug implant. Stated alternately, “lesion” refers to a specific area of “damage” (as broadly defined above, which can include weakened or irregular surfaces) having a size which makes that area suited for replacement by a plug implant, regardless of what caused the damage, and regardless of the grade or severity of the damage or chondromalacia at that site.

### Femoral Runner Repair

Although the plug implant holders described herein can be adapted for use in joints other than knee joints (and, for use in animals other than humans), the primary focus for the discussion, descriptions, and drawings herein, is on plug implants designed to repair femoral runners, in knee joints. There are several reasons for that choice of focus, including the following reasons, initially as a short list, followed by more extensive discussion below:
i. The need for simpler, faster, and less expensive repairs to damaged cartilage on femoral runners offers, by far, the greatest and most frequent and common “unmet need”, in the field of orthopedic surgery.
ii. It can offer severe, difficult, and complex challenges and obstacles, in orthopedic surgery.
iii. If a set of plug implants designed and sized for replacing damaged lesions in femoral runners can be shown to perform successfully and effectively, then any such plug implants can be directly adapted and modified to enable these types of plug implants to be used to repair other cartilage segments, including tibial plateaus, and for repairing damaged hyaline cartilage in hip, shoulder, elbow, ankle, and even finger and toe joints.

Accordingly, because of the combination of factors briefly listed above, and because of the significant need for better devices and methods for repairing knee joints in patients who truly need better help (and better options than the ones currently available), the Inventor herein (whose entire career was as a full-time orthopedic surgeon) chose to focus (beginning roughly 20 years ago, and proceeding continuously throughout that entire time span) exclusively on new types of devices and methods for repairing knee joints. The research he has done (or collaborated in), to date, has focused on better ways for repairing damaged cartilage in knee joints. The discussion herein – except for occasional passing references to other types of joints – represents and describes the research he has done, and the insights he has gained, in work that has focused on primarily knee joints.

To provide additional information, cartilage damage on femoral runners requires surgical intervention and repair more commonly, and frequently, than any other type of cartilage-repair surgery. That high level of frequency arises from a combination of factors, including the following: (i) the knee ranks alongside the shoulder, as the two most complex and complicated joints anywhere in a mammalia body. In knee joints, that complexity arises from the complex surface shapes of the femoral runners, the tibial plateau, and the patella (kneecap), which must all interact, not just with each other, but also with meniscal cartilage, and with a complex set of tendons and ligaments; (ii) the femoral runners are very near the surface; indeed, the “medial” runner can be felt directly, through the skin, by anyone who rubs a finger against the surface of a knee, just inside and slightly below his or her kneecap, while the knee is fully bent. That unprotected position, directly below the skin, makes femoral runners much more vulnerable to injury than other cartilage surfaces in “weight-loaded” joints, most of which are surrounded and protected by deeper and thicker layers of muscles, tendons, and other tissues; and (iii) the knees are: (a) subject to heavy compressive weights and loadings, while standing; (b) subject to even higher compressive loadings while walking, running, or jumping (including “peak loadings” which can be more than double the “static load” that occurs when a person is standing on one leg); (c) subject to large lateral forces, whenever a person or animal is walking on a sloped or uneven surface; and, (d) highly vulnerable to lateral blows, which can come from any direction.

Because of those and other factors, the hyaline cartilage on femoral runners becomes damaged, and must be surgically repaired, more frequently any other segment of cartilage, anywhere on the body.

Two crucial and central problems which have hampered prior efforts to create good and useful “plug implants”, for replacing only a targeted lesion on a femoral runner while leaving the remainder of that cartilage segment and unchanged, can be described in the two points listed directly below: (i) dislodging and displacement of prior “plug implants” that were being tested occurred in substantial numbers of cases; and, when that happens, it can lead to excruciatingly painful crises, and to severe damage inflicted on other bones and tissues in the knee joint; and, (ii) in prior attempts at a type of plug implant to repair only a targeted area on a femoral runner, the edges and discontinuities around the plug (which are unavoidable whenever a “plug” implant is attempted, arising directly from how the phrase “plug implant” is defined) pose major risks, over a span of months or years, of scraping against, abrading, and inflicting serious damage on the “opposing” cartilage segment, which the repaired femoral runner must press against and slide across, when someone is walking or running.

As to the second point listed above, a crucial type of abrasion and damage that has been caused, by previously tested versions of plug implants in femoral runners, was not inflicted on the cartilage which surrounds that plug implant, on that same femoral runner. Instead, it was inflicted on a “tibial plateau” segment of cartilage, which sits on the top surface of the tibia (i.e., the long bone inside the lower leg).

Since many prior “plug implants” for femoral runners have not been truly successful, nearly any type of surgical repair of seriously damaged cartilage (such as cartilage torn in an injury or accident, and any cases of chondromalacia which have reached Grade 3 or 4) on femoral runners usually requires (if not initially, then within a span of roughly ten years, in nearly all cases) the type of surgery called a “total knee replacement” (abbreviated as TKR). That type of surgery requires the lower end of an entire femoral bone to be sawed off, and replaced by a hard and rigid metal implant having very smooth, highly-polished metal runners at the bottom end. After the TKR surgery, those metal runners will slide and move across a hard plastic segment which must be inserted, to replace the “tibial plateau”; and, that requires the entire top end of the tibial bone to also be sawed off, and entirely replaced.

Also, many well-done TKR’s additionally require replacement of the kneecap (patella) as well. TKR’s which do not also replace the kneecap may tend to suffer from high failure rates, which are often severe enough to require a second major surgical operation, within ten years or less after the first TKR surgery.

To make things even worse, TKR surgeries suffer from a major set of problems and risks. Patients continue to suffer from serious problems, even after they have had TKR surgery. Pain issues, as well as feelings and/or episodes of instability that are serious enough to make a TKR recipient feel unsteady, uncertain, and afraid, even when walking on a flat or just mildly sloping or irregular surface, are so common that nearly any orthopedic surgeon who has been practicing for ten years or more likely has encountered multiple patients who actively and unhappily complain that they are not satisfied with the results of a TKR operation they underwent, even after more than six months, which is long enough for a satisfactory outcome to settle in and stabilize, if that is going to happen.

Accordingly, many of those problems can and would be avoided, in large numbers of patients (especially among the elderly, the frail, and certain other categories of candidate patients who need some relief, and who will not subject a repaired knee joint to frequent bouts of heavy and/or demanding usage), if simple and convenient “plug implants” were available, for knee repairs in those types of patients. Rather than subjecting someone in their seventies (and who no longer aspires to take long hikes in the mountains) to a total knee replacement, a simple “plug implant” to repair a specific damaged spot, in a knee joint, could be performed within less than two hours (indeed, less than a single hour is an entirely feasible goal, for a skilled surgeon who has become experienced and comfortable with the devices and methods used), on an outpatient basis, without requiring general anesthesia or an overnight stay in a hospital; and, it could enable a convalescence period of less than a week, compared to the multiple weeks (and sometimes months) that must pass before many TKR patients feel that their artificial knee joint has reached some level of normalcy, stability, and usefulness.

### Adaptation to Other (Non-Femoral) Hyaline Cartilage Repairs

As mentioned above, if “plug implants” can be created which are able to meet all of the difficult demands and challenges of repairing damaged cartilage on femoral runners in knee joints, then similar implants, having essentially the same design as disclosed herein, but with sizes and shapes that can be modified and adapted for other types of joints, can be easily developed, for repairing damaged cartilage in other joints.

For example, injured or arthritic fingers and toes often require replacing a relatively small, generally circular, disc-shaped hyaline cartilage segment between two adjacent bones in a finger or toe. Even though such cartilage-replacing implants in “the digits” are not subject to the types of loading stresses that occur in knee joints, there is a general need for improved “button”-shaped implants for repairing damaged cartilage in finger or toe joints. Accordingly, even though the designs described and illustrated herein were developed for difficult and demanding uses in knee repair, they can be adapted, easily and directly, to be sized and shaped for implantation into injured, arthritic, or otherwise damaged or diseased finger or toe joints.

In addition, the types of plug implants disclosed herein also can be adapted for repairing cartilage in shoulder joints. In humans, the cartilage segment attached to the shoulder blade (i.e., the “scapula”) is not a true “socket”; instead, it is called the “glenoid cavity”, and it is shaped somewhat like a small and shallow tray, with a diameter not much larger than a quarter (i.e., the American coin).

In addition to enabling complete replacement of the “glenoid cavity” on a shoulder blade (scapula), using the same type of “plug implant” implant holding device described herein, these types of “plug implants” can also be adapted, in at least some cases, to replace specific targeted areas of damaged cartilage on the large rounded “head” of the “humerus” (i.e., the long bone in the upper arm), in shoulder joints.

Similarly, these types of “plug implants” can be adapted for replacing a limited-size area of damaged cartilage, on either the rounded and generally ball-shaped “femoral head”, or the “pelvic socket” (also called the acetabulum, or the acetabular socket), in a hip joint. If tissue imaging (using X-rays, ultra-sound, MRI’s, etc.) indicates that a pain-causing cartilage defect in a hip joint is limited to a single specific area, on either or both of the “ball” and “socket” surfaces, then that/those specific area(s) of damaged cartilage can be replaced by a plug implant.

Surgical methods and devices for performing that type of surgery, and for securing an implant to the surrounding bone at the exact desired and intended location (i.e., not by approaching the surface from outside the bone, but by drilling an access tunnel through the bone, from below the surface). While possible use in human hip joints will require extensive testing and clinical proof before it can be approved, it could be developed for use on dogs with hip problems, and on large livestock animals, in a fairly simple, rapid, and inexpensive manner.

Despite the above-listed options for use in other types of joints, the focus of the description and figures herein (and, the research that has been done to create the present disclosure, and to prove that these devices can and will function as intended) is on developing the best possible types of plug implants for repairing damaged cartilage on femoral runners. That specific type of use poses a challenge, and the need which cannot be adequately satisfied by any prior devices or methods.

### CRITERIA FOR “PLUG IMPLANTS” THAT ARE RELEVANT TO THE PRESENT DISCLOSURE

Several criteria can be regarded as significant to cartilage-repair “plug implants” such as the current invention.

First, as mentioned above, these implants are directed to “plug”-type implants (as mentioned above, these also can be called partial implants, spot implants, button implants, or similar terms), for replacing hyaline cartilage. Although these types of implants can be modified and adapted, to enable them to entirely replace a small segment of cartilage (such as in a finger, thumb, or toe joint), “plug” implants as described and claimed herein are not designed to replace an entire segment of native cartilage; instead, the term “plug” implies that they necessarily will share at least one edge with a piece of undisturbed native cartilage; and, in most uses (especially on femoral runners, which will be one of their most common uses), a plug implant will be fully surrounded by undisturbed native cartilage.

Second, they must be designed for “permanent” surgical implantation. In this context, “permanent” indicates that an implant has no fixed or intended limit on how long it will remain in place, after it is surgically implanted. In elderly or frail patients, any such implant hopefully will endure – remaining fully functional, and not causing any significant pain or discomfort for the patient – for the entire remaining life of the recipient. In younger patients, they should endure for enough years, and hopefully decades, to render such implants truly useful, helpful, valuable, and appreciated, even if a patient decides to have a “total knee replacement” after some span of time.

The third main limitation arises from a factor which requires some explanation, due to the fact that surgical implants for repairing hyaline cartilage can be divided into two entirely different and non-overlapping categories.

One category includes bio-regenerating implants, which also are sometimes called “cartilage-regenerating implants”, “tissue engineered (or engineering) implants”, or similar phrases. They also are sometimes called resorbable implants, even though only a single component will be resorbable, since the “resorbable” material they contain is crucial to how they function. The term “resorb” refers to a process in which at least some quantity of previously solid material will be gradually dissolved, and liquefied, via digestion of a biological or biocompatible material due to cellular activities, in a way which causes the resorbable material to dissipate, disappear, and have its molecular components broken apart and then re-incorporated into a new and different solid material, such as newly-generated cartilage. As one example, “old” collagen proteins are constantly being recycled and replaced by “new” collagen molecules; and, almost all cell types other than neurons are constantly being killed, recycled, and replaced by new and vigorous cells, by a programmed cell-killing-and-recycling process called “apoptosis.” Rather than being destructive or painful, that process of constant turnover, recycling, and replacement, of proteins and cells, is what enables mammals and other large animals (and their muscles, organs, tendons, ligaments, blood vessels, and other soft tissues) to remain vigorous and healthy for more than just a few months or years. Accordingly, “resorbable” implants contain at least one component (usually in the form of a matrix, or mesh) which is made of a resorbable material (usually processed collagen fibers) which will actively encourage certain types of cells (usually called mesenchymal and/or chondrocyte cells) to move in and occupy that mesh or matrix, and begin creating and secreting new collagen fibers, in the hope that the new collagen fibers will form new cartilage having a shape, thickness, and structure that are very similar to the prior hyaline cartilage in that location, before it became injured or diseased.

The second (and entirely different and non-overlapping) category includes mechanical devices, which also are called non-resorbable devices. Instead of trying to enable and promote the creation of new cartilage, these devices attempt to use a manufactured synthetic material to “permanently” (as defined above) replace a damaged segment of cartilage.

The differences between those two approaches, and the types of devices and materials they use, are central and crucial to how they function, and what they try to accomplish. Accordingly, any “bio-regeneration” efforts (and any implants which contain a significant quantity of a “resorbable” material that is designed to dissolve and disappear as it is being replaced by newly-formed cartilage) are not relevant to this current invention, which is limited solely to “mechanical” implants (aka “non-resorbable” and/or “permanent” implants).

Another relevant term which people working in this field sometimes use, and often encounter, is “biomimetic.” Laypersons often use it in a fairly broad and non-specific way, to indicate that some component of a permanent, mechanical, non-resorbing device will somehow “mimic” (i.e., emulate, resemble, etc.) the properties of the bone or tissue which it is replacing. However, when experts use it, they usually intend to imply that a material was chosen and selected (or created and developed, if necessary) to closely resemble the cellular and molecular structure, as well as the mechanical, behavioral, and performance traits, of the biological thing which it is intended to replace. Stated in alternate terms, a biomimetic material, or device, is a material or device which carefully and intelligently draws inspiration from nature, and from the materials and processes used by nature to solve complex biological problems.

Accordingly, although some might erroneously take the position that a “non-hydrous” (or “non-hydrated”) polymer can be biomimetic, when compared to natural cartilage, if it has stiffness and flexibility levels similar to cartilage, that is not how the term is used by experts, and it is not how that term is used herein. The motion and travel of free and unattached water molecules, within natural cartilage, is a crucial, critical, central feature in how natural cartilage performs, and behaves, and a non-hydrous polymer cannot mimic or emulate that crucial feature, because it simply does not contain enough free water molecules to affect its physical properties and behavior. By contrast, hydrogel polymers can be biomimetic, if enough of their other properties also resemble the properties of natural cartilage, because their chemistry and structure was inspired and guided by a drive to create something which truly resembles, not just how cartilage flexes, but how cartilage is structured, and how it responds to localized pressures in ways which intimately involve and depend upon the motion and travel of free, unattached water molecules through the polymer strands which make up the matrix of the cartilage.

### ANCHORING SURFACES WHICH PROMOTE “BONE INGROWTH”

Certain comments above – which describe the differences between: (i) implants designed to regenerate cartilage, and (ii) implants designed to permanently replace cartilage – refer only to the articulating surfaces of such implants, i.e., to the smooth and lubricated material surface which will press and slide against a different segment of hyaline cartilage which covers the surface of a different bone, in a joint. The articulating surface of a cartilage repair implant alternately can be called an exposed, outer, upper, or sliding surface, or similar functional terms.

By contrast, those comments do not also apply to the anchoring surface of a cartilage-repair implant. That phrase refers to an implant surface that will be pressed directly against a hard bone material, in a manner which will seek to solidly and securely anchor any such implant to the bone it will be mounted on.

Certain technology used to create “anchoring surfaces” for cartilage repair implants, for any “large joint” (such as a knee, hip, shoulder, etc.; there may be some exceptions, for some types of implants designed for “small joints”, such as in fingers or toes) uses a combination of: (i) some type of initial means (such as screws, pins, etc.) for securing an implant in place, affixed to a bone surface with enough strength and durability to keep it stationary for a span of at least several weeks, to provide enough time for hard bone material to “grow into” a layer of porous ingrowth material on that anchoring surface, without the implant being dislodged, which would severely disrupt or even destroy the bone ingrowth process; and, (ii) bony-tissue ingrowth into the porous layer on the anchoring surface of the implant, in sufficient quantity to securely attach the implant to the bone it was affixed to, in ways that will keep the implant permanently affixed to that supporting bone (hopefully, for the remaining life of the patient, so that the only way it can be dislodged is if a major and severe traumatic injury somehow dislodges the implant from the bone).

The anchoring surfaces of some cartilage-repair implants used a specialized metal alloy which was manufactured in a way that created porosity in an otherwise rigid and very hard bone-compatible material. In the past, some implants used alloys containing titanium; however, in some of the more recent types of implants, a polymer called PEEK (for poly-ether-ether-ketone; it can be formulated in ways which make it very hard and durable) is used, instead of a metal alloy.

These types of anchoring surfaces usually were made by “mashing” together (under very high manufacturing pressures, in the range of tons per square inch) extruded “strands” (which are readily visible, with diameters usually about half a millimeter or so) of a chosen alloy, into a desired shape, while the metal strands are hot enough to make them malleable. That manufacturing process will create a “nest” of mashed-together metal strands, with small gaps and pores between them. In modern times, other methods which fall into the categories called “3D printing” or “additive manufacturing” (discussed below) can be used to create similar porous surfaces that will promote bone ingrowth; accordingly, either type of approach (as well as “hybrid” approaches, which can use, for example, low-cost mass-manufacturing methods to create an intermediate “workpiece”, which can then have porous bone-ingrowth layers added to its surface) can be used to make the types of implant holding devices described herein, from either metal alloys, or polymers such as PEEK.

During surgery, a surgeon will use one or more tools (usually drill bits) to deliberately dig into a bone surface that an implant is being affixed to. That step is done to a controlled and limited level, to activate any cells embedded within that bone material, and trigger them into launching an active repair response. Surgeons usually refer to that as “refreshing” a bone surface.

Subsequently, after the surgery, during the weeks and months of healing, the bone-generating cells which are directly pressing against the porous anchoring layer of an implant will reproduce, and will grow into the vacant spaces between the 3D printed porosities or mashed-together strands of metal, and will begin secreting the hard crystalline bone material called “apatite”, in the same way that cells adjacent to a bone fracture will generate new hard bone material, when they need to repair a fractured bone.

The next critical area is the smooth and lubricated “articulating” surface of any new type of implant, and on how the articulating surface component is designed, and supported by the other components of an implant; and, in this specific discussion, attention must focus on the hard and rigid holding component which is designed to securely hold a flexible polymeric insert (preferably made from a hydrogel polymer) in a way that will allow the rigid holder, and the flexible insert, to work together, to create a “plug implant” for replacing a lesion of damaged hyaline cartilage.

Accordingly, a discussion is provided below of the main types of flexible materials which offer the best candidates for use as described herein. In one of the principal (and most probable) manufacturing pathways, these types of materials can be injected (under pressure) into an implant holding device as described and claimed herein, while in a viscous liquefied precursor form; then, that precursor liquid will “set” (also called curing, hardening, solidifying, or similar terms) into a solid but flexible form, to finish the manufacture and assembly of a “complete” plug implant. The term “complete” (when used to refer to an implant device) is used herein to refer to a device which has both a hard implant holding device as described herein, and a flexible insert which is permanently affixed within the implant holding device, regardless of whether that device might be subjected to one or more subsequent steps, often referred to as polishing, finishing, or quality control steps.

### THE CHEMISTRY AND PERFORMANCE OF POLYMERS AND HYDROGELS

One of the key features and benefits of devices and systems of the present disclosure is that testing has shown that a “hydrogel”, as described herein, when placed in the type of “container” device having the anchoring and reinforcing components and arrangement described and claimed below, has: (i) performed in entirely satisfactory ways, which closely emulate the behavior of natural hyaline cartilage, in joints, in large test animals, such as sheep; and, (ii) also have been shown to be remarkably and even extremely durable, when the types of “plug implants” described herein have been tested, using the types of wear and abrasion tests required by the Medical Devices division, within the US Food and Drug Administration.

Hydrogel materials, as a group, have a high ability to emulate, mimic, and match the behavioral traits and performance of natural cartilage better than known plastic, polymer, metal alloy, or other known solid material (as a brief reminder, all references herein to “cartilage” are limited to hyaline cartilage, and exclude elastic cartilage, and fibro-cartilage). This arises directly from the fact that natural cartilage is itself a hydrogel; and, as such, it evolved in ways that render it ideally suited to “articulate” in the smoothest and most durable ways possible, when it presses against another hydrogel material, in a closed environment that contains and is lubricated by “synovial fluid” (i.e., the fluid that all vertebrate animals create and use, as a wetting agent and lubricant for cartilage surfaces, inside articulating joints).

Several terms are discussed below, which relate to the types of “solid but flexible materials” that can emulate the performance and behavior of hyaline cartilage, and which therefore offer candidates for use in “plug implants” for replacing lesions of damaged cartilage on femoral runners.

As a starting point, the term “polymer” is usually given a simplified definition, as “a large molecule which is assembled from small building-block molecules, called monomers.” That definition needs to be accompanied by a crucial additional factor: to create a true polymer, the building-block molecules need to be assembled in a specific controlled and desired manner, to create large molecules which will be desirable, functional, and useful, rather than creating mere “agglomerations” (which also can be called aggregates, accretions, or similar terms) that do not have a desired molecular structure, and which cannot perform in desired ways.

It also should be noted that, under the definition above, proteins, DNA, and RNA all qualify as “biological polymers.” Proteins are formed by stringing together amino acids, as their monomers, and DNA and RNA are formed by stringing together molecular subassemblies called “nucleotides,” as their monomers. Accordingly, any references below to a flexible “polymeric” material or insert component include and cover flexible polymeric materials that are derived from proteins, which in most cases will be (or will include, as a major component) a specific type of protein called “collagen.”

Collagen requires a bit of focused attention, because it is the most important and abundant (by far) structural fibrous protein, in vertebrate animals. In nearly all cases, it is secreted by cells, and needs to be recognized and regarded as an extra-cellular protein; and, in nearly all cases, it is formed and secreted in a manner that creates “bundles” out of the long individual molecules, in a manner directly comparable to how strong ropes are created by bundling together large numbers of thin fibers.

In the body of any vertebrate animal, most of the collagen forms an “extra-cellular matrix”, which any and all “anchorage-dependent cells” (as distinct from blood cells) attach to, as they form any type of “soft” or “connective” tissue (as distinct from bones). Also, bones contain small amounts of collagen, in ways that help establish their shapes and structures, but bones are formed mainly from minerals which provide hardness, via a crystalline “lattice” which combines negatively-charged atoms (mainly oxygen and phosphorous) with positively-charged atoms (mainly calcium), in a molecular arrangement that is usually called apatite, or hydroxyapatite (which, like collagen, is created and then secreted by specialized cells, to form its own special type of extracellular matrix).

By contrast, the elongated strands of the protein molecules called collagen do not have a crystalline structure. Instead, they are flexible, in ways that make them ideal for creating the “scaffolding material” that cells adhere to, to form “soft tissues”, such as muscles, internal organs, tendons, ligaments, the walls of blood vessels, etc.

Collagen is also the protein which forms cartilage, and gives it shape, structure, strength, and its own specialized behavioral traits and properties. Indeed, it forms all three types of cartilage (as listed in the opening paragraphs of this application), including hyaline cartilage (the only type of cartilage of which is of interest herein).

Hyaline cartilage contains a few cells, interspersed within the matrix of aligned collagen fibers which make up the cartilage; however, they are limited to small numbers, since a large number of cells (which are surrounded by permeable membranes which can be ruptured and torn apart by even modest tensile and/or shearing forces), if dispersed throughout cartilage, would seriously weaken the cartilage.

Rather than being a compacted and hard protein material (comparable to fingernails or animal claws, which are made from a very different extra-cellular protein, called keratin), cartilage has a surprisingly high water content, which in most cases is slightly more than 60% by volume. That water content consists of free, unattached water molecules, which can readily and rapidly squeeze and travel through the collagen fiber bundles which create the cartilage “matrix”, in response to localized pressure differentials on a cartilage segment such as a femoral runner (as occur during the cyclic knee motions that occur when a person or animal is walking or running).

That type of gel-type “water-containing flexibility” is a crucial, essential, central characteristic of natural cartilage; and, no known water-free synthetic polymer has ever been discovered, despite numerous efforts to do so, which can adequately emulate and come close to matching all of the various different performance and behavioral traits of cartilage, in satisfactory ways, in a flexible plastic material which is sufficiently durable to enable a cartilage-replacing implant to last for multiple years, in a “weighted” joint such as a human knee. Accordingly, researchers have focused on creating or identifying some type of polymer which can be used to provide cartilage-replacing implants which can be used to repair cartilage in knee joints (which, as described above, must grapple with loadings, stresses, and other challenges that are much more difficult than occur in any other joint, and which must be surgically repaired, more frequently than in any other joint), in ways that can provide “plug implants” (as distinct from the hard plastic components which will entirely replace a sawed-off set of femoral runners, as used in “total knee replacements”).

Accordingly, the term “hydrogel” needs to be defined, since cartilage is a type of hydrogel. A hydrogel is a “bi-phasic” material which contains both:
(i) large numbers of “free” water molecules, where the adjective/prefix “free” indicates that they are not attached to any other molecules, and instead, are free to travel and move within and through the long molecules which form the “matrix” that will give the hydrogel material its shape and strength; and,
(ii) a “matrix” created by long strand-like molecules, which can be either synthetic polymers or biological proteins, and which are bonded, inter-twined, entangled with, and/or otherwise interconnected and/or “coupled” to each other.

In order for any particular type of bi-phasic “hydrogel” to qualify as a potential candidate for use in a “plug implant” intended for implantation in a femoral runner in a human knee joint, the matrix of polymer or protein molecules, in any such candidate hydrogel, should be able to give that candidate hydrogel sufficient strength, and durability, to perform, for multiple years, as a hyaline cartilage substitute, in a weighted joint such as a human knee joint, in a large adult male (and, indeed, in a seriously overweight large adult male, since excess weight is a direct contributing factor to the types of knee damage that often require surgical intervention).

A potential goal of a truly well-designed and optimal plug-implant holding component (such as described herein) is to provide enough support, against different types of force and stress which will be imposed on the flexible component of an implant (which will include compressive forces, lateral forces, torsional (i.e., rotational) forces, and shearing forces), to enable that flexible component to not just survive, but to remain unaltered, unaffected, undamaged, and unscarred, despite years of use, even in a large and seriously overweight male adult. That is one of the goals of the implant holding devices described herein.

Past implant designers and makers have had apparent difficulties in trying to use various hydrogel materials to create plug-type implants for use in weighted joints such as knees, and have focused, instead, solely on plastic-type polymers that are slightly flexible, but which are not bi-phasic, or biomimetic (as described above), and do not contain any free water molecules.

However, the fact remains that hyaline cartilage is, itself, a “hydrogel”; and, because of how it evolved, over hundreds of millions of years, it is ideally adapted and suited to rub and press hard against an “opposing” surface made of another hydrogel.

Therefore, a holder is needed, designed so that it will give enough support to a true hydrogel material, in a surgical implant, to help make that hydrogel strong enough, and durable enough, to give multiple years of service, when implanted on a femoral runner, in a knee joint. The implant holding devices of the present disclosure satisfy such a need. Even though these implant holding devices can be adapted and used, if desired, to hold and support non-hydrogel plastic-type polymers, one of the main design goals throughout this entire research project has been to create implant holding devices, for “plug implants” on femoral runners, which can fully enable the use of bi-phasic hydrogel materials in such implants.

Returning to the subject of polymers and polymer chemistry, the molecules which form the matrix of a hydrogel are indeed polymer molecules, since no other types of molecules can provide a combination of long enough, strong enough, and “interactive enough” (via cross-linking bonds, entanglements, and other types of couplings, as described below) to create a matrix that can be strong enough and durable enough to function as the flexible component of a plug implant for a femoral runner. Therefore, the difference between “polymers” versus “hydrogels” depends, not on the chemistry of the long matrix molecules, but on whether that polymeric matrix contains enough “free water” content to make it a truly “bi-phasic” material, where free water molecules – i.e., unattached water molecules which can readily flow and travel through and between the long polymeric molecules of the matrix – are present, in a quantity which is able to create and constitute its own “phase” of the material, in a way which will be able to substantially alter the physical and mechanical properties and load-bearing behavior of that material.

Among synthetic polymers, there are numerous contributing factors which should be recognized by those wanting to better understand why some particular polymer will behave and perform in a certain way, or who is trying to select the best polymer for some particular use, or trying to create an even better polymer, by adapting and tweaking some known and promising starting candidate, to see how various possible modifications will affect its properties and behaviors.

One factor focuses on the long “backbone” chains that are created, when monomer building blocks are bonded to each other. The lengths of those chains can be controlled, by means such as controlling the temperature and duration of a polymerizing reaction, and/or by putting a controlled concentration of a specialized “chain-terminating” monomer reagent, into a batch of chain-forming monomers.

Another factor involves the question of whether non-carbon atoms should be included directly within the backbone chains, and if so, what atoms should be used, and what their spacing should be. As just one example, the polymers that may be commonly referred to as “nylon” is actually an entire class of polymers, containing nitrogen atoms inserted directly into their backbone chains, with numerous different variants. To gain a brief glimpse into the variations between different types of nylons, any non-expert is encouraged to do an internet search to find out the differences between nylon-6, versus nylon-66, which are made from different reagents, and have substantially different performance traits; indeed, each of those designations refers to a “family” of subtly different formulations. It is safe to assume that polymer candidates containing any and all types of non-metal atoms which will form two or more bonds have already been tested, in various attempts to create new and useful polymers, and research articles describing that type of research can be found in the nearly 20 different journals which are listed below, all of which are devoted to polymer chemistry.

Another major set of factors involves “pendant side groups”, which can be attached to the backbone chains of a polymer. The exact combination of atoms that will be included, in any such “side groups”, has major and even profound effects on how any specific polymer will perform; and, the spacing(s) between any such side groups also provides another set of controllable factors. In addition, there are ways to incorporate two or more (or even multiple) different types of side groups (having different lengths, and/or different atoms and groups) into synthetic polymers; as just one example of how that can be done, two, three or even more monomeric reagents can be mixed together, in a “starting batch” of reagents.

Among other things, the “side groups” attached to the polymeric “backbone” molecules will contribute heavily to whether any particular polymer will be “hydrophilic” (which means that the side groups will actively attract water molecules into the polymer), or “hydrophobic.” Most of the commercially important synthetic polymers (which includes the categories that most non-experts simply call “plastic”) are strongly hydrophobic, so that they will repel and resist water, rather than being softened and potentially weakened by it.

Yet another major set of factors involves the question of whether single atoms will be attached to atoms that are directly in the backbone chain of a polymer. For example, oxygen, nitrogen, or sulfur atoms can be double-bonded to a carbon atom in a backbone chain, in ways which can strengthen and stabilize a polymer. An example can be seen in any drawing of the molecular structure of polyurethane, which has a double-bonded oxygen, attached to a carbon atom which is adjacent to a nitrogen atom, in the backbone chain. Pictures of urethane bonds, in backbone chains, can be found quickly and easily, via an internet search.

Yet another entire set of major factors involve the presence, spacing, density, and lengths of crosslinking attachments (often called bridges, connectors, linkers, crosslinkers, or similar terms) between different backbone chains, in a polymer. Carbon atoms will form hard materials only under certain conditions, and in certain types of molecular structures (with diamonds and graphite as examples). In most other types of polymeric materials, each and every carbon atom will form four bonds, with four other atoms; and, since bonds between atoms are formed by paired electrons, all of which have negative charges, the four bonds formed by each and every carbon atom will stay as far away from each other as they can. As a result, the classic angle between each of the four different bonds, in the classic “tetrahedral” shape of a molecule of methane (CH4, which looks like it has a triangular base made of three of the hydrogen atoms, with the fourth hydrogen atom pointing upward, away from the other three hydrogen atoms) is about 110 degrees; and, that same lowest-energy, most-stable bond angle occurs in nearly all “single bonds” that involve carbon atoms. That angled arrangement creates a zig-zagging (rather than straight and linear) molecular structure, when carbon atoms are coupled together in a chain. Therefore, “hard” plastics can be formed by creating multiple closely-spaced and relatively short and stiff “cross-linking” bridges or connectors, between different backbone chains in a polymer, and by additional steps to make a certain plastic material even harder (such as including nitrogen and/or oxygen atoms and/or double bonds in the polymerized molecules). Conversely, softer and more rubbery and “elastic” polymers can be created by spacing the “cross-linking” bridges or connectors farther apart from each other, and/or by making them longer, and more flexible.

Yet another factor which will affect how any specific polymer will behave arises from the question of whether the molecular structures which attach different backbone molecules to each other will be formed by covalent bonds, hydrogen bonds (or other “ionic attraction” bonds), or “side-group entanglements.” Each of those factors can be controlled, and a polymer can be provided with some combination of several different types of “cross-linking” attachments. Also, most professional chemists limit the term “crosslinking” to attachments that use “covalent” bonds, which are the strongest types of bonds between any two atoms; however, not everyone uses that term consistently, and some people will refer to any molecular “linkage” which somehow attaches or couples one polymeric backbone molecule, to a different polymeric backbone molecule, as a “cross-linking” attachment.

Still more “major factors” in polymer chemistry involve the use of “plasticizers” and other specialized reagents, which (like “chain-terminating” monomers) can be added, in controlled concentrations, to a blend of reagents that will be used to make a polymer.

It also should be noted that polymers can be provided with “bonding agents” which will enable those polymers to act as adhesives, which can directly and tightly adhere (or “stick”, attach, affix, or similar terms) to various types of solid surfaces.

In light of the foregoing comments, and as a result of the factors described therein, two inter-related points are significant to the present disclosure, as follows:

First, because hyaline cartilage is itself a hydrogel, and because of how it evolved, and how it is ideally adapted and suited to rub and press hard against a surface made of another hydrogel (i.e., the “opposing” segment of cartilage hydrogel) in an articulating joint, therefore, in any surgical implant which is designed to repair a single cartilage surface, in a knee joint, and which (after the surgery) will rub directly against an unaltered surface of native hyaline cartilage (i.e., against another hydrogel material) in that repaired knee joint, a “plug implant” having an “articulating surface” made of a hydrogel, can and will be able to perform better than any type of “plug implant” having an articulating surface made of a polymer or plastic which is not a “hydrogel”; and,

Second, testing has shown that, by providing a “container device” (as described below) which is ideally suited for enclosing, supporting, and anchoring a hydrogel component in a “plug implant”, this invention discloses, for the first time, a method of designing “plug implants” which will be strong enough, stable enough, and durable enough, to provide truly workable, useful, and durable “plug implant” repairs for knee joints, which contain hydrogel materials as their articulating surfaces.

The two points asserted directly above are a significant aspect of the present disclosure, and it is preferred that the “articulating surface” of any such cartilage-repair “plug implant” described or claimed herein should be made of a hydrogel material, containing a substantial quantity of free water molecules (when measured by either weight, or volume, as discussed in more detail below).

The present disclosure describes and claims implant holding devices, designed for use as plug implants in femoral runners, which are designed to hold, and which are fully capable of holding, any of the above-listed types of flexible materials, as the cartilage-emulating material in a plug-type implant. These devices can be tested and evaluated, for possible use in plug implants for femoral runners, when filled by any flexible polymeric or proteinaceous material that is of interest to any researcher, any research team or group, or any orthopedic supply company. Therefore, the present disclosure does not depend upon, and does not recommend, endorse, or teach, the selection and use of any particular type of flexible polymer (whether a hydrogel, or a non-hydrous polymer), over any other candidate flexible polymer. Instead, it simply discloses and claims a biocompatible implant holding device made of a hard and non-flexible material (such as a titanium alloy, or a PEEK or similar polymer which is formulated to be hard, to a point of rigid), which is believed to be capable of improving the strength, durability, utility, and value of any such plug implant, after any selected flexible polymer material has been loaded into this implant holding device, compared to any other previously-known candidate implant holding devices.

### SHAPE AND PHYSIOLOGY OF FEMORAL RUNNERS

Several aspects of the present disclosure can be better understood by appreciating several terms that are used by orthopedic surgeons, when they discuss femoral runners and certain other aspects of knee joints.

One set of terms arises from the fact that, when viewed from either side, femoral runners have a shape which is roughly circular, but which deviates from being truly circular, in a controlled manner. That shape is referred to as a “cam” shape, and it is somewhat similar to the shape of a chicken’s egg, when seen from an angle that shows its non-circular shape (anyone who is not familiar with how “cams” work should find and study a picture of a “camshaft”, as used in automobile engines to control the timings of the openings and closings of the fuel, air, and exhaust valves, for each cylinder in the engine).

FIG. 11 provides a simplified view of the shape of a femoral runner, shown from the side, with notations added to show five different “zones” which have different levels of curvature. The terms “anterior” and “posterior” have their conventional meanings, to refer to the front side, and the back side, respectively. The location between them, which is aligned with the main shaft of the femur bone, is called the “distal” zone (or tip) of the femoral runner. The two zones called “mid-anterior” and “mid-posterior” cam zones also are shown in FIG. 11.

Several of those zones (i.e., all of them except for the “posterior” zone) can be directly accessed by an orthopedic surgeon, using minimally-invasive surgical tools, when a patient is lying down, on his/her back (often called a “supine” position) on an operating table or chair, with the knee joint flexed (bent) to whatever level is necessary, and held stationary by a combination of supports and straps, which are standard components of the types of tables and chairs used for knee surgeries. These types of surgeries can be performed without requiring general anesthesia (i.e., the types of anesthesia which renders a patient unconscious), by using localized and/or regional anesthesia, such as used in well-known types of “spinal block” or “epidural” anesthesia, to prevent a patient from feeling anything that is happening in one leg (or sometimes both legs).

A “posterior” intervention requires a different indirect approach. Damage to the posterior zones of femoral runners is rare, and those types of operations are rare; nevertheless, they sometimes occur, and surgeons can use “indirect” instruments (as described in a separate patent application by the same Inventor here) to implant a “plug implant” into the posterior zone of a femoral runner, in a specific patient, if a need arises.

Accordingly, the “plug implants” of the present disclosure can be manufactured in a range of sizes and shapes, which are described below, since they are part of the present disclosure. When an orthopedic surgeon assesses a patient, the main factors of interest will be the height of that patient, whether that patient is male or female, which of the five “zones” on a femoral runner needs to receive a plug implant, and the size and extent of the damaged “lesion” (the largest diametric dimension will provide the crucial number). Once those factors are known, and have been confirmed by X-ray, ultrasound, or a minimally-invasive visual inspection, the surgeon will know the size and shape of the implant that will be needed, for the surgery, and can order that device, with delivery available only a day or two later, in most cases. And, if a specialized implant is needed with one or more non-standard dimensions, that can be accomplished in only a day or two, since computer-controlled “additive manufacturing” (often called “3D printing”), as described below, can be used to make these devices, and it is a simple and convenient task to insert a new set of dimensions into a computer program, which will then control a “3D printing” device as it makes a custom-ordered implant having a specified set of dimensions.

To optimize the implant/host bearing surface match of an implant to the patient, the surgeon must ascertain the anatomic location of the cartilage defect being replaced and confirm the optimal match with the trocar drill guide. FIG. 12 provides further illustrations of implants 10 arranged in the femur 2 and the tibia 4.

### ADDITIVE MANUFACTURING ( AKA 3D PRINTING)

Several candidate methods of “3D printing” or “additive printing” have been developed, and refined to advanced levels, which have moved this field of art beyond the range of working with meltable plastics and/or polymers to make things, and into creating metal alloys, usually from powdered (particulate or granular) feedstock materials; and, it should be noted in particular that the types of hard metal alloys that can be made via 3D printing include titanium, which is used in various “biocompatible” alloys that have been used for decades, in devices such as hip or knee implants.

It may also be noted that “additive manufacturing” can be used to add additional layers or components, to components or devices that were machined or otherwise mass-manufactured, using methods that can be faster and cheaper than using printers to add only a few microns at a time to something that is being manufactured.

Accordingly, anyone interested in the use of “additive manufacturing” to make precision pieces from hard metal alloys should be aware of each of the following processes, as candidates for that type of manufacturing:
(1) “direct metal laser sintering” (DMLS), which uses a laser beam to heat a metal powder to a temperature which will fuse the particles together, without fully melting them;
(2) “selective laser melting” (SLM), which uses a laser to fully melt the metal atoms in a powdered feedstock, in ways which can create materials that are denser and harder than can be created by sintering;
(3) “electron beam melting” (EBM), which functions in a manner similar to “selective laser melting”, but which uses an electron beam, instead of a laser beam, to reach the desired temperatures. This method requires vacuum chambers that can achieve very high vacuum levels, so each electron beam machine can create only a single item (or a very limited number of items) at a time, and it is more expensive than other options. However, due to the same “high vacuum” parameters that increase its cost, EBM can create metal pieces that are denser and harder than laser melting, and is therefore worth considering, for manufacturing surgically implantable items that will need to last for years in high-stress environments such as knee joints.

In addition to the above, laser beams can be used to “carve out” and remove specific targeted area of metal, from a metal piece. This is similar to the way that ornamental devices, and devices having names, company trademarks or logos, etc., programmed into the pattern, made of laser-carved wood, are manufactured. While that process does not fall properly within the field of “additive manufacturing”, it functions in similar ways, and offers another option for manufacturing one or more layers or components of an implant holding device as described and claimed herein.

### DIRECTIONAL TERMS USED HEREIN

The final item that needs to be addressed, in this Background section, refers to how various directional terms are used herein. Clarity on these terms is necessary, because these devices will be manufactured in one orientation, and then implanted in any of several different orientations, none of which resemble the manufacturing orientation. Therefore, as described below, all directional terms used herein refer to the orientation that will be used during manufacture.

To avoid confusion, all directional terms that are used herein (such as top, bottom, above, below, etc.) will refer to a single, consistent orientation of one of these implant holding devices. As a general overview, the present disclosure discloses an “open bowl” type of device, made of a generally hard material, which is designed to be filled with an “insert component” made of a flexible material which can emulate cartilage. For reasons described below, the most likely and probable type of manufacturing operation will involve injecting, into the holder, a viscous liquefied precursor compound, and then causing or allowing that precursor liquid to cure (or set, harden, solidify, or similar terms) into a flexible solid material, within the holder. When that manufacturing step (or a possible alternate approach, in which a pre-manufactured flexible piece is force-fit into an accommodating holder, as discussed below) is carried out, it will create a “complete” plug implant which will include both the hard holding component (as described and claimed herein), and a flexible cartilage-emulating component that has become firmly and securely embedded (or affixed, installed, or similar terms) within the holder. The phrase “complete implant” is used to refer to a holder-and-polymer combination, regardless of whether any additional polishing, finishing, inspection, quality control, or other manufacturing steps are subsequently performed on a “complete” device.

Accordingly, the holding component of such an implant can be regarded as having a miniaturized “open bowl” shape and orientation, with the base on the bottom, and the opening on the top. In that orientation, the anchoring surface (which will be pressed directly against a hard bone surface, and which will be a porous material which promotes bony tissue ingrowth into that anchoring surface) will be on the bottom side, and on the lower portions of the cylindrical walls of a sidewall.

By contrast, the articulating surface (which will become the smooth, slippery, lubricated surface that will press, slide, and rub against an “opposing” cartilage surface) will be on the top side.

Accordingly, any directional terms used herein (such as top, bottom, upper, lower, higher, below, etc.) will use that orientation as a reference point, and will assume that the implant holding device is sitting in a resting position, on a table, counter, or other horizontal surface, like a conventional bowl which is ready to receive and hold a batch of soup, cereal, or similar content.

### SUMMARY OF THE INVENTION

Aspects of the present disclosure disclose an implant holding device, made of a generally hard and in most cases rigid material, which is designed and suited to be filled with a flexible polymeric compound, such as a hydrogel, which can emulate the traits and behavior of hyaline cartilage. When combined, an implant holding device and a flexible insert will create a “plug implant,” which will be designed, sized, and suited for permanently replacing a lesion of injured, diseased, or otherwise damaged cartilage on a femoral runner, in a knee joint, without changing or disrupting any adjacent or surrounding native cartilage, and without requiring “total knee replacement” surgery.

Cartilage repairs on femoral runners are among the most frequent types of orthopedic surgery, and yet, no one has ever previously been able to create “plug implants” which can perform adequately. Over a span of more than a decade of sustained and ongoing research in this field, the Inventor herein (an orthopedic surgeon) gradually assembled enough insights and ideas to create a “complete plug implant” device which can reach and even surpass that goal; and, the implant holding devices which can be used to create such plug implants comprise a separate invention in its own right (which also offers a powerful example of the close interplay between form, and function). Accordingly, it is described and claimed by specifying each and all of: (i) the forms of the various components; (ii) the optimal assembly of those components; (iii) the functions which these implant holding devices can achieve, when those parts have been assembled or fabricated in an optimal arrangement and then filled with a suitable flexible polymeric compound (preferably a hydrogel compound), to create a “complete” plug implant. Brief mentions are also included, describing certain types of new and innovative tools (and methods for using those tools), which had to be developed, to enable successful surgical implantation of “complete” plug implants as described herein, into damaged femoral runners.

Accordingly, aspects of the present disclosure (and the claims below) focus on the implant holding device. In addition to being manufactured and sold in the form of “complete implants” having flexible insert components, these hard and rigid holdings devices also will be manufactured and sold on a “stand-alone” basis (i.e., without containing any polymeric compound), to any qualified research group, company, or agency, to enable any such qualified researcher, company, or group to load any type of flexible polymeric compound that they wish to test and evaluate, into copies of these implant holding devices, so that they can then test how well that particular candidate polymer can perform, in a plug-type implant, either for repairing damaged femoral runners (which is one of the most difficult and daunting challenges in orthopedic surgery), or for repairing or replacing any other segment of hyaline cartilage, in any other type of joint, in humans, pets, or livestock.

As a brief summary of the components, these implant holding devices have a generally “miniaturized open container” configuration, with a base and a peripheral sidewall, both of which have porous anchoring layers on a portion of their external surfaces (to promote the ingrowth of bony tissue into the “anchoring surfaces” of these implants), as well as a “gripping surface” on their internal surfaces; a barrier layer 38, positioned inside those components, to prevent the liquefied polymer precursor from reaching and clogging the porous anchoring surfaces; and a bottom vent, to allow a gas or liquid to escape from the bowl structure, while being displaced by a liquid that is being injected (under pressure) into the bowl structure.

In addition, for any implants which are designed to hold a hydrogel material as the articulating surface, one or more reinforcing components can be positioned inside the bowl structure, such as in the form of one or more struts (or ribs, cross-members, or similar terms) that will traverse that open space, in a way which can strengthen the hydrogel without creating a risk of creating gas-filled vacancies in the final hardened hydrogel polymer.

Furthermore, the upper surface of the sidewall will allow this type of plug implant to be forcefully driven into a prepared vacancy (which has been drilled and machined into the supporting bone), by an initial “firm tapping, using a mallet” procedure, followed by gentle tapping (to provide precise control over the final depth of the implant in the hard bone) when the implant approaches its exact desired depth (which, in most cases, will be flush with the surface of the hard bone that is being repaired). In addition, a slight taper may be provided on the outer surface of the implant holding device, with each side being about one degree away from the centerline. As a result, when a tapered implant is forcibly driven into a cylindrical bone vacancy created for the implant, the tapered outer surface will act in a manner comparable to a wedge being driven into a gap, in a way that will enable the implant to gain a stronger and better initial fixation to the hard bone material which surrounds that prepared insertion receptacle.

An array of these types of implants may be made, having a range of diameters that can be used to replace lesions of essentially any size that is suited for replacement by a plug implant; and, these implant holding devices can be used to create “complete implants” containing polymer components that have been molded into a “surface radius of curvature” that will allow a mass-manufactured implant to be selected and ordered, which will have a specified diameter, and a specified surface radius, that will enable any such implant to fit smoothly into place, during surgical implantation, in a way that will closely align its shape and surface, in all directions, with the undisturbed surface of the surrounding or adjoining native cartilage.

This type of “open bowl” device can provide an ideally balanced combination of both:
(i) lateral support, for an upper layer of hydrogel material which will extend slightly above the upper edge of the sidewall in the implant, even within the highly demanding setting of a plug implant on a femoral runner in a knee joint; and,
(ii) a sufficient “open flow” layer, in the thin layer of hydrogel material which sits above the upper edge of the sidewall, to allow rapid and responsive travel of free water molecules and synovial fluid between the “hydrogel plug” and the natural undisturbed cartilage which surrounds that “hydrogel plug”, to closely emulate and mimic the behavior of a normal, natural, undisturbed hyaline cartilage surface, despite the fact that a portion of that surface has been surgically altered, to replace a segment of injured, diseased, or otherwise damaged hyaline cartilage, with a plug of a synthetic hydrogel material sitting inside a metallic bowl-type device.

In an aspect, it is an object of the present disclosure to facilitate arthroscopic joint resurfacing of bone-on-bone synovial joint pathologies using a hydrogel, before progressive bone erosion manifests to cause joint deformities indicating more drastic treatment is necessary.

In another aspect, the present disclosure is directed to fixedly securing a flexible material such as a hydrogel to a bone overcoming one or more problems associated with the existing solutions, as well as providing additional features which address the issue of total joint replacement and open surgery being the common approach to cartilage repair and replacement.

The present disclosure provides for an improved anchoring system to deliver and securely anchor a hydrogel medical device to replace damaged cartilage in a mammalian joint, using an anchor having a hollow or shelled out interior, such as an internal cavity, to optimize the secure fixation of the bone anchor to the cartilage replacing hydrogel to enhance the longevity of the connection between the anchor and the hydrogel being anchored. The present invention also discloses how an anchor according to an aspect of the present disclosure can be assembled in an array to support a larger sheet of hydrogel to resurface an entire joint surface.

Another object of the present disclosure is to provide improved methods of stable secure fixation of a soft polymer or hydrogel bearing surface to a relatively rigid bone recipient site establishing a modulus of elasticity gradient from the rigid bone to the compliant bearing surface, resulting in a stable replacement device for damaged cartilage in an arthritic joint.

Another object of the invention is to provide a method of securing a rigid bone anchor to a relatively flexible cartilage replacement bearing surface by creating a hollow/shelled out anchor which accommodates an extension of the soft bearing surface into the bone anchor, resulting in an enhanced fixation of the connection of the anchor to the softer hydrogel, and enhanced performance of the thickened hydrogel.

Another object of the invention is to provide a method to disclose a method of securing a larger sheet of hydrogel with an array of hollow/shelled out anchors with extensions of the hydrogel into the hollow/shelled out anchor enhancing the connection between the cartilage replacement hydrogel/polymer and the array of bone anchors.

Another object of the disclosure is the delivery and secure, stable anchoring fixation of a hydrogel or polymer bearing surface into a synovial joint for replacing damaged cartilage in mammalian joints.

Another object of the disclosure is the delivery and secure, stable anchoring fixation of a hydrogel or polymer bearing surface into a synovial joint for replacing damaged cartilage in mammalian joints, such that host recipient site tissue healing occurs in such a manner as to engage the device anchoring mechanism.

Another object of the present disclosure is to reduce the stresses experienced at the implant bone interface through the rigid anchoring of the device to the recipient bone.

Another object of the present disclosure is to increase the longevity of the composite gel and the hollow/shelled out medical device anchor during physiologic function.

Another object of the disclosure is to reduce the stresses, during normal physiologic joint motion, experienced by cartilage, of the articulating joint surfaces of the femur and tibia thereby reducing arthritic wear of the cartilage of these surfaces.

Another object of the disclosure is to reduce the stresses, during normal physiologic joint motion, experienced by implantable devices to replace damaged cartilage, of the articulating joint surfaces of the femur and tibia, of the knee, as well as the articulating surfaces of other bones forming the articulating surfaces of other mammalian synovial joints, thereby increasing the longevity of these implants.

The present disclosure provides, in an aspect, a hydrogel medical device is fabricated with an integral anchor having an anchor that is hollow and/or shelled out with a cavity into which hydrogel flows during a molding process and fills the bone anchor cavity while within the mold. This molding process of the hydrogel onto and affixed within a portion of the bone anchor results in the permanent fixation of the hydrogel to the anchor. The molded device can be delivered into an arthritic joint through a minimal incision. Once delivered, the device can be installed with secure, stable fixation of the bone anchor to rigid bone tissues at the implant recipient site and thereby securing the hydrogel bearing surface to the bone to replace the damaged cartilage surface. The molding flow and extension of the hydrogel into the bone anchor cavity provides that the hydrogel is securely affixed and gripped by the anchor. The anchor is fixed rigidly to bone and thereby fixes the hydrogel rigidly to bone.

It is another object of the present disclosure to facilitate arthroscopic joint resurfacing of bone-on-bone synovial joint pathologies using a hydrogel, before progressive bone erosion manifests to cause joint deformities indicating more drastic treatment is necessary.

The present disclosure further provides, in another aspect, for a bone anchoring system with an improved gel anchor interface coupled with an improved anchor bone interface, to securely fix the lubricious load bearing hydrogel directly to bone through a layered 3-D printed anchor interface.

The methods disclosed herein can, in certain aspects, employ a 3-D printed anchor of the proper shape, size and thickness for securely fixing all or at least a portion of a meniscal segment.

The present disclosure further provides, in another aspect, an anchor that has at least a portion porous enough to permit the passage of a flexible material for replacing cartilage such as a hydrogel into the pores of an adhesion or attachment layer, during the molding process. The anchor further includes a barrier layer 38 to prevent the flow of the hydrogel into the pores of a bone healing layer of the anchor that is generally positioned on an opposite side from the adhesion layer. The adhesion layer may preferably be designed with pores greater than 0.4 mm for accepting a hydrogel during molding, to enable the anchor to securely grip the hydrogel replacement layer for replacement of the damaged cartilage. The porosity may be determined by a lattice design (such as a lattice defined as the 3-D spatial orientation of the array of struts connected to create a latticework or array of connected struts) for the hydrogel adhesion, strut (strut defined as the individual structural member assembled together to create a lattice) thickness between 0.2 and 0.6 mm, which determine the structure of the lattice, the thickness of the gel adhesion layer between 1.5 mm and 3 mm and the space between the struts, the actual pores (pores defined as the empty spaces between individual or collection of struts in a lattice). An anchor according to the present disclosure may also be designed with a bone healing layer for permanent bone healing, with pores sized between 0.15 mm and 0.45 mm. The strut thickness (0.2-0.6 mm), “trabecular” (trabecular referring to the orthopedic term used to describe bone anatomic architecture) lattice structure, layer thickness (min 0.5 mm) and pore size (0.15-0.5 mm) to optimize bone healing. Restriction of hydrogel flow from the hydrogel adhesion layer to the bone healing layer can be accomplished with a change in the porosity at the junction of the gel adhesion and bone healing layer, creating a barrier layer 38, to prevent the flow or passage of hydrogel across this barrier junction. Alternatively, a solid barrier layer 38 can be incorporated into the design, such as in the file for a 3-D printing process, with overlap between the gel adhesion and the bone healing layers into the solid barrier layer 38. This design increases the strength of the three layers printed together, with overlap of the layers, preventing any void weaknesses, thereby enhancing implant anchor strength. This overlap enhances to implant anchor strength from the gel adhesion layer, through the solid barrier/binding layer, to the bone healing layer.

Digital additive manufacturing (3D printing) is advantageously used to form anchoring structures of metal or other materials, wherein the anchoring structures comprise small but intricate internal supporting lattices, and are incorporated together with a molding barrier layer 38, during an additive manufacturing process employing plural materials, finally being infused with hydrogel during a molding process that results in a durably joined hydrogel surface, merged into a lattice (up to the barrier layer 38) and a bone growth layer joined to the lattice.

Another object of the present disclosure is to securely anchor to bone a hydrogel prosthetic cartilage to replace damaged cartilage, with a 3-D printed anchor with one layer of a pore size greater than 0.4 mm for a hydrogel infusion, resulting in secure adhesion of the hydrogel to the anchor with the hydrogel facing one exposed surface of the cartilage replacement. The anchor has at least one further layer, preferably formed integrally with the hydrogel-infusion layer but the further layer has a pore size between 0.15 mm and 0.5 mm for bone tissue healing, facing the opposite exposed surface. In an advantageous arrangement, a barrier layer 38 blocks infusion of the hydrogel into the further layer during molding. This implant can be embodied in or formed as a press fit plug, a threaded implant to be screwed into the bone, as well as secured with adjunct staples, pins and screws.

Another object of the disclosure is to provide a relatively thin anchor with a layered construction of variable porosity to enhance gel adhesion and bone healing with a barrier layer 38 to prevent gel flow from the gel adhesion layer into the bone healing layer during the molding process.

Another object of the present disclosure is to reduce the stresses experienced at the hydrogel interface between the anchor and the gel to enable long term functional survival of the implant.

Another object of the present disclosure is to increase the longevity of the composite gel, and 3-D printed medical device during physiologic function.

Another object of the disclosure is to reduce the stresses, during normal physiologic joint motion, experienced by cartilage, of the articulating joint surfaces of the femur and tibia thereby reducing arthritic wear of the cartilage of these surfaces.

Another object of the disclosure is to reduce the stresses, during normal physiologic joint motion, experienced by implantable devices to replace damaged cartilage, of the articulating joint surfaces of the femur and tibia thereby increasing the longevity of these implants.

In one aspect, the present disclosure is directed to an implant for use in a medical procedure. The implant includes a body and a flexible material. The body includes a perimeter sidewall having an upper free edge, an outer surface, and an inner surface, and a bottom wall (or base), connecting the sidewall on one edge, having an inner surface and an outer surface. The body also includes a cavity defined by the inner surfaces of the sidewall and bottom wall (or base), and a top opening into the cavity bordered by the upper free edge. The flexible material is connected to the body and is positioned in the cavity and has at least a portion extending out of the top opening.

In another aspect the present disclosure is directed to another implant for use in a medical procedure. The implant includes a plurality of anchors, each including a body. The anchor bodies each include a perimeter sidewall having an upper free edge, an outer surface, and an inner surface, a bottom wall (or base) having an inner surface and an outer surface, a cavity defined by the inner surfaces of the sidewall and bottom wall (or base), and a top opening into the cavity bordered by the upper free edge. A flexible material is connected to the plurality of anchors and is positioned in the body cavities and extends out of the top openings. The flexible material forms a pad having a bottom surface which covers the plurality of anchor top openings.

In another aspect, the present disclosure is directed to a medically-implantable anchor for attaching a flexible material to a bone. The anchor includes a bone attachment layer having a porous structure for facilitating attachment to a bone via bony ingrowth, a flexible material attachment layer having a porous structure for receiving the flexible material and helping to retain the flexible material to the anchor, and a barrier layer 38 sandwiched between the bone attachment layer and the flexible material attachment layer and being impermeable by the flexible material. The bone attachment layer forms a first surface of the anchor and the barrier layer is shaped as a container to include a bottom wall (or base) and a sidewall extending away from the bottom wall (or base). Inner surfaces of the bottom wall (or base) and sidewall define a cavity configured to receive the flexible material, and the flexible material attachment layer is positioned in the cavity.

In a further aspect, the present disclosure is directed to a method of manufacturing an implant for use in a medical procedure. The method includes providing a body. The body includes a perimeter sidewall having an upper free edge, an outer surface, and an inner surface, a bottom wall (or base), connecting the sidewall on one edge, having an inner surface and an outer surface, a cavity defined by the inner surfaces of the sidewall and bottom wall (or base), and a top opening into the cavity bordered by the upper free edge. The method further includes attaching a flexible material in the cavity, at least a portion of the flexible material extending out of the top opening.

In yet another aspect, the present disclosure is directed to a method of manufacturing an implant for use in a medical procedure. The method includes providing a plurality of anchors each having a body. The anchor bodies each include a perimeter sidewall having an upper free edge, an outer surface, and an inner surface, a bottom wall (or base) having an inner surface and an outer surface, a cavity defined by the inner surfaces of the sidewall and bottom wall (or base), and a top opening into the cavity bordered by the upper free edge. The method also includes attaching a flexible material to the plurality of anchors by positioning the flexible material in the body cavities such that a portion of the flexible material extends out of the top openings, the flexible material forming a pad having a bottom surface which covers the plurality of anchor top openings.

In yet another aspect, the present disclosure is directed to a method of treating a cartilage disorder in a synovial joint. The method includes preparing the site for receiving an implant and providing the implant. The implant includes an anchor having a body. The body includes a perimeter sidewall having an upper free edge, an outer surface, and an inner surface, a bottom wall (or base), connecting the sidewall on one edge, having an inner surface and an outer surface, a cavity defined by the inner surfaces of the sidewall and bottom wall (or base), and a top opening into the cavity bordered by the upper free edge. The implant further includes a flexible material affixed to the anchor with at least a portion in the cavity. The method further includes affixing the implant to a bone at or adjacent the implant site such that the flexible material is positioned to act as cartilage in the synovial joint.

### BRIEF DESCRIPTION OF THE DRAWINGS

The foregoing Summary and the following detailed description will be better understood when read in conjunction with the appended drawings, which illustrate a preferred aspect of the present disclosure. In the drawings:

FIGS. 1A and 1B are perspective views of an exemplary medical implant, according to an embodiment of the present disclosure;

FIGS. 2A and 2B are cross-sectional views of an anchor of the medical implant of FIGS. 1A and 1B;

FIGS. 3A and 3B are cross-sectional views of the medical implant of FIGS. 1A and 1B;

FIGS. 4A and 4B are cross-sectional views of a medical implant and anchor according to another embodiment;

FIG. 4C is a top perspective view of the embodiment of an anchor of the medical implant of FIGS. 4A and 4B;

FIG. 5 is a cross-sectional illustration of an embodiment of a medical implant according to the present disclosure;

FIGS. 6A-6C are cross-sectional views of a medical implant and anchor according to another embodiment of the present disclosure;

FIGS. 7A and 7B are cross-sectional views illustrating an assembly configuration of a medical implant according to the present disclosure;

FIGS. 8A-8C are perspective views of an anchor showing a layered structure according to an embodiment of the present disclosure;

FIGS. 9A-9C are perspective views of another anchor showing an alternative layered structure according to an embodiment of the present disclosure;

FIG. 10 is a schematic illustration showing relative pore size for a layered anchor, consistent with aspects of the present disclosure;

FIG. 11 illustrates femoral runner cam zones and representative radii of curvature;

FIG. 12 provides an illustration of implants arranged in the femur and tibia;

FIG. 13 illustrates an implant having a flexible material defining a radius of curvature;

FIG. 14 is a flowchart of an exemplary manufacturing process for a medical implant, consistent with aspects of the present disclosure;

FIG. 15 is a flowchart of another exemplary manufacturing process for a medical implant, consistent with aspects of the present disclosure; and

FIG. 16 is a cross-sectional illustration of a molding assembly for forming a medical implant, consistent with aspects of the present disclosure.

### DETAILED DESCRIPTION

As summarized above, aspects of the present disclosure comprise a generally bowl-shaped implant holding device, which may be formed as a container, made of a hard material such as a titanium alloy, which is designed and suited to receive and hold an insert component made of a flexible polymer or protein material which can emulate cartilage (such as a bi-phasic “hydrogel” having sufficient “free water” content to make it truly bi-phasic). When a flexible insert is inserted into the type of rigid implant holding device disclosed herein, the combined components will create a “plug implant” which can be used to replace a segment of damaged hyaline cartilage on a femoral runner, without having to replace an entire segment of cartilage, and without requiring “total knee replacement” surgery. As described and defined above, a “plug” implant must share at least one border with (i.e., be directly adjacent to) an undisturbed segment of native cartilage; and, in many and probably most cases of femoral runner repair, a plug implant will be entirely surrounded by undisturbed native cartilage.

As also described above, these types of plug implants can be referred to as either “mechanical” or “permanent”, since they do not attempt to promote any regeneration of new cartilage. In nearly all cases, they will be intended to remain in place for the entire remaining life of a recipient. However, if desired, they can be used as “bridging”-type devices, to provide a limited number of months or years of service, as a part of a planned multi-step procedure which will extend out over a span of months or years (such as, for example, if cartilage regeneration methods and materials improve to a point where a mechanical plug-type implant device can enable a patient to have near-normal knee functionality, on an interim basis, while new cells and cartilage are being grown in cell culture conditions).

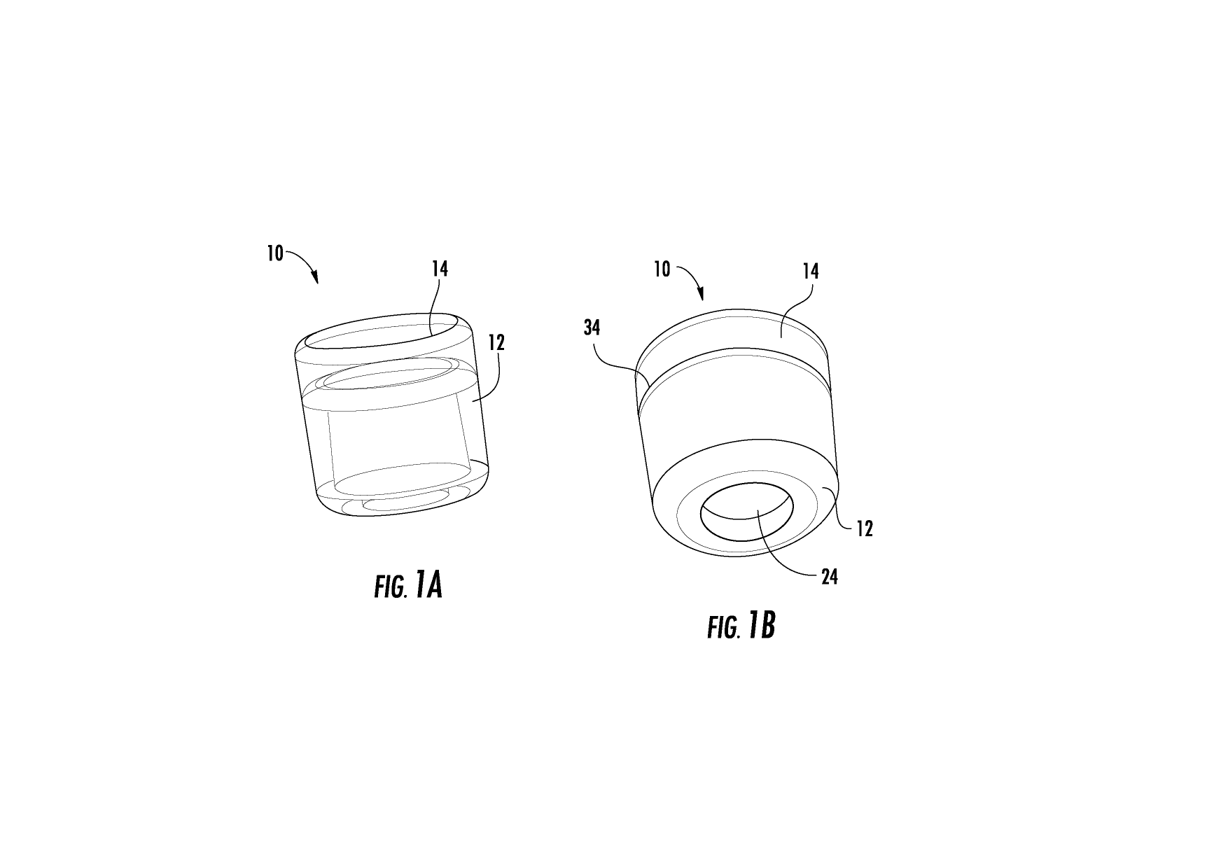

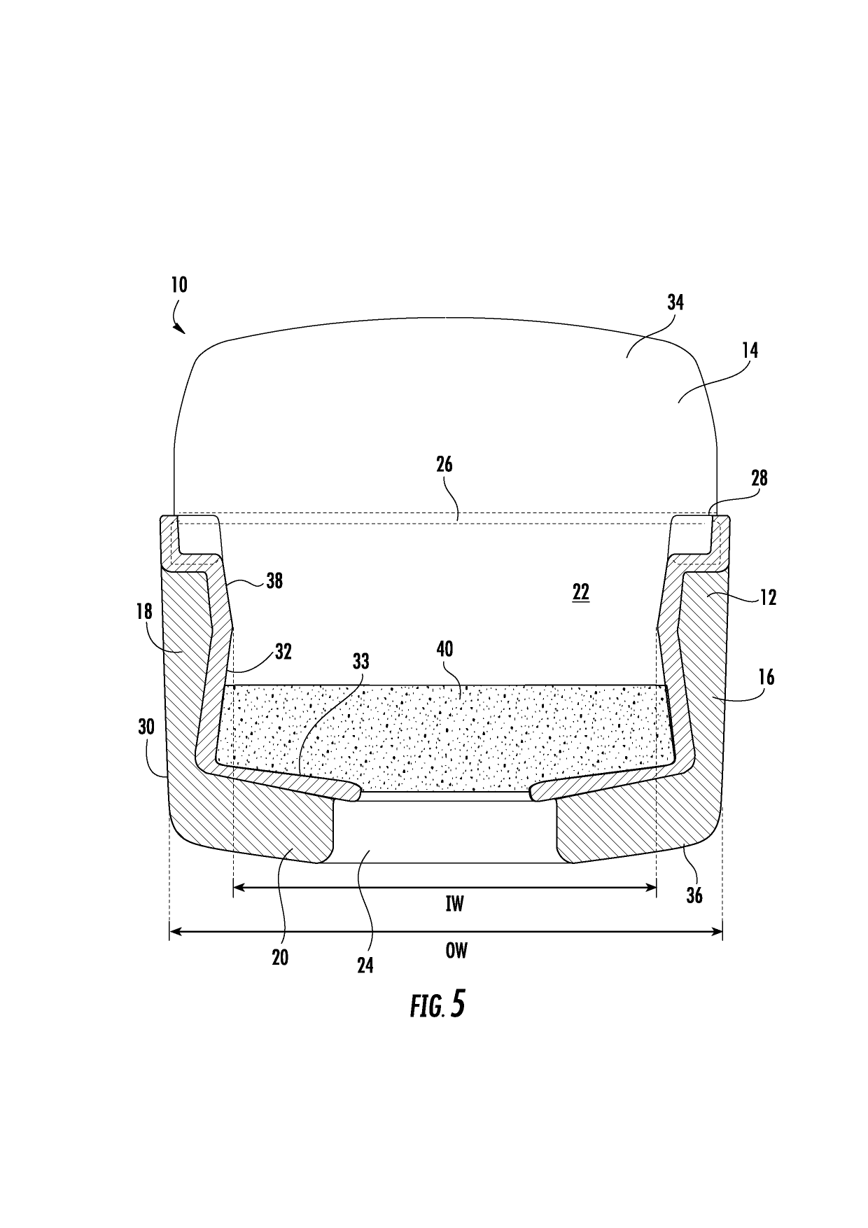

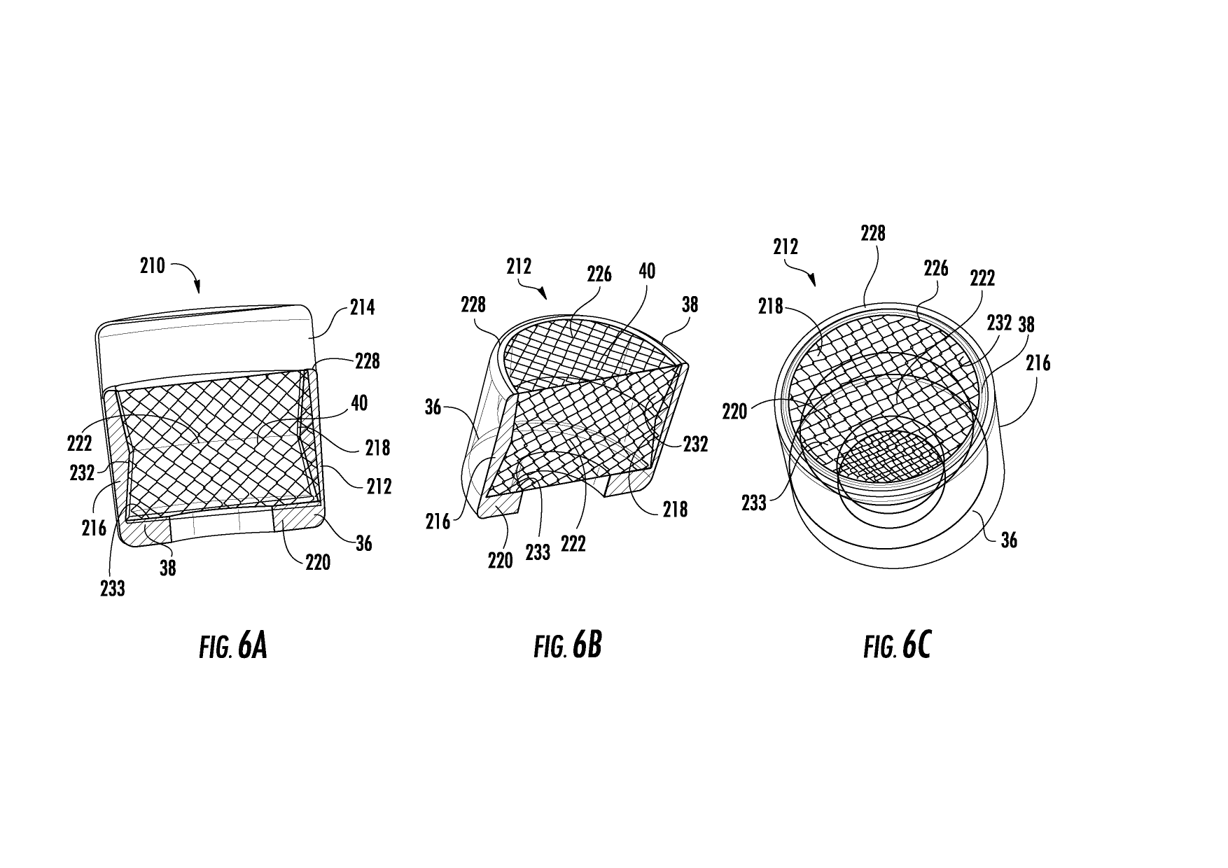

Accordingly, FIGS. 1A-10 and 13 are depictions of various embodiments of plug-type implant holders 10, 110, 210, 310 (also referred to as implant holder devices or implant devices) as disclosed herein. Distinctions between these various embodiments are outlined in greater below. As shown in the Figures, the implant holders 10, 110, 210 comprise a container body 12, 112, 212, 310 having a sidewall 18, 118, 218, 318 and a base 20, 120, 220, 320.

The sidewall 18, 118, 218, 318 forms a perimeter of the container body 12, 112, 212 and comprises at its top an upper edge 28, 128, 228, which may also be referred to as a “rim.” The sidewall 18, 118, 218, 318 surrounds a cavity 22 comprising an interior area of the container body 12, 112, 212. The upper edge 28, 128, 228 comprises an upper portion of the sidewall 18, 118, 218, 318, and faces away from the base 20.

The sidewall 18, 118, 218, 318 may be a generally cylindrical wall, which will provide mainly lateral support for the flexible cartilage-emulating material that will be molded or otherwise inserted into the holder, in a “complete implant” as that phrase is used herein.

A flexible material 14 is held within the implant holders 10, 110, 210. The flexible material 14 is formed to act as the new, supplemental or replacement cartilage and includes properties which are consistent with such use. The flexible material 14 is preferably a flowable material that is capable of being injection molded and at least partially solidified, while still retaining a level of elasticity, to form a cartilage-like surface on a bone. In an exemplary embodiment, the flexible material 14 is a hydrogel material, such as described above. The flexible material 14 may be formed from one or more hydrogels having different properties. For example, a soft hydrogel may form a base of the flexible material 14 for attachment to the anchor as further described herein, while a thickened outer hydrogel is configured to act as a cartilage-like replacement. The flexible material 14 may also be a polymer, such as polycarbonate urethane, or other material having similar properties, as would be known to a person of skill in the art.

In the embodiments shown in the Figures, the implant holders 10, 110, 210 will have a generally circular shape, when viewed from above, partly so that no complications can be created by a rotational orientation being out of a desired alignment, and partly so that an implant can be inserted into a “bone vacancy” (i.e., an open hole which will serve as a receptacle and holder for the plug implant) which can be drilled by a conventional drill bit. However, after surgeons become familiar and comfortable with installing circular plug implants, other types of plug implants can be created which will have alternate shapes, such as oval or elliptical shapes, to replace a larger “lesion” (i.e., a specific limited area of damaged cartilage) having a shape that cannot be replaced adequately by a circular plug. Bone vacancies having specialized shapes, to accommodate non-circular implants, can be created by using a grinding bit to enlarge an initial circular hole, with the aid of a template, a computer-controlled manipulator, or any other suitable device or system. To make oval, elliptical, or custom-sized plug implants even more useful, any such implant can be fabricated or assembled in a way which will make its “articulating surface” (i.e., the smooth upper surface, which (in hydrated and lubricated form) will press, rub, and slide against an “opposing” segment of cartilage, on the surface of a different bone) have any specific shape that is necessary to enable that specific implant to perfectly “match up” with (and fit smoothly into) the surface contours of the undisturbed cartilage surrounding the area of damaged cartilage which will be replaced by a plug implant.

Considering circular-shaped plug implants, a range of plug implants having three different diameters (12 mm, 16 mm, and 20 mm) may be made, as a “baseline” set of implants that will be able to handle most knee repairs that are suited for surgical repair by using a plug implant.

However, it should also be appreciated that for each of the standard diameters, a range of different “surface radius of curvature” implants for each specific size implant also will be available, as options. In simple terms, the “surface radius of curvature” parameter (shortened below to “surface radius”, since “radius” directly implies “curvature”) indicates the amount of “rise”, in a dome-shaped surface, compared to an imaginary flat plane established by the outer rim of the dome.

The term “dome” and its variants (such as “dome-shaped”) refers to a desired and intended surface shape, which will necessarily rise above (in a smooth and slightly rounded manner) a surrounding rim, so that the articulating surface of the flexible insert, in a plug implant, will be the only surface that an “opposing” cartilage segment (which covers the surface of a different bone) will contact, and rub and slide against. If that type of “dome” shape is not provided properly, the hard upper edge 28, 128, 228 would scrape against, abrade, and gradually damage the opposing cartilage segment. In certain types of plug implants disclosed herein, the upper edge 28, 128, 228 may be flat (i.e., “planar”), to simplify installation, minimize any risk of sub-optimal placements, and keep manufacturing costs (per unit) down. However, after surgeons become accustomed to working with these new types of plug implants, other alternative shapes can be created, including oval or elliptical shapes as mentioned above, and even shapes which can be custom-specified for a single individual patient. Accordingly, the term “dome” as used herein is not limited to only circular domes; instead, it also can be applied to the types of controlled and desired gradually-rounded shapes that can rise up, slightly, above the level of an oval-shaped, elliptical-shaped, or other type of rim.

If a dome has a small “radius of curvature”, that number will indicate that the highest center portion of the dome will be substantially higher than the surrounding rim surface; and, by contrast, a large “radius of curvature” will indicate a lower, more gradual dome. To help anyone visualize and understand this trait, consider what someone would obtain if they cut circular discs, having the same 1-inch widths, from each of a golf ball, a baseball, and a soccer ball. The dome-shaped disc from the golf ball – with the smallest “radius of curvature” – would be very round, and would extend upward about half an inch, in the center, if set on a table with the flat side down. The dome-shaped disc from the baseball, with the middle-sized “radius of curvature”, would be less round, and would extend up about a quarter-inch; and, the segment from the soccer ball would have only a small dome shape, about 2 millimeters high. In a similar manner, if an implant holder having a fixed diameter (such as 16 mm) is filled with enough flexible polymer to create a sizable dome in the middle, then that dome will have a small surface radius of curvature. If the same-sized holder is filled with enough polymer to create a smaller but readily noticeable dome in the middle, it will have a middle-sized radius; and, if it is filled with only enough polymer to create a low and barely noticeable dome in the middle, it will have a large radius.

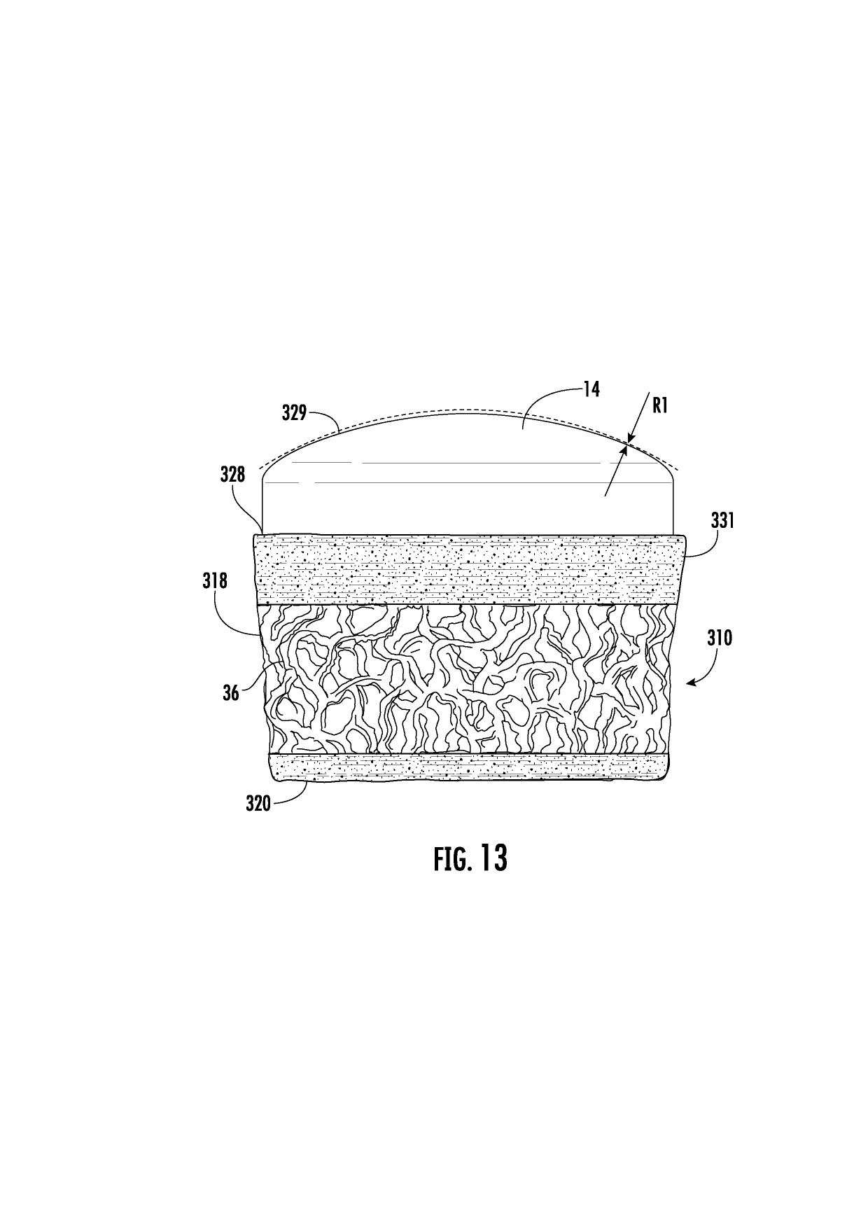

FIG. 13 illustrates an implant 310 according to aspects of the present invention, comprising a sidewall 318, a bottom wall 320, an upper edge 328 at the top of the sidewall 318, and a flexible material 14 held in the implant 310. In the illustrated example of FIG. 13, the flexible material 14 having a top surface 329 defining a radius of curvature (R1). This radius of curvature (R1) is configured to match, conform to, correspond to, or mimic a patient's anatomy, i.e. a curvature of a patient's femur in a region of the implantation site. In one aspect, the radius of curvature (R1) can be in the range of 15 mm-40 mm.

While some specific diameters (i.e., 12, 16, and 20 mm) are mentioned, it must also be recognized that any such holder, having any diameter, can be filled (by using specialized custom-developed “molding shells” during the manufacturing operation) with an amount of polymer which will create an exact desired “surface radius of curvature” for a flexible insert which will be held within that implant holding device; and, the “radius of curvature” chosen for a specific implant will render that implant ideally suited for repairing a femoral runner lesion that will need repair, in a specific patient. The exact sizes and shapes of the dome-shapes inserts is discussed below, under the section heading, “Size Ranges for Standard-Sized Circular Implants.”

Accordingly, any surgeon who is planning to implant a plug-type implant in a specific location, on a damaged femoral runner in a specific patient, will assess the size and location of the lesion(s) in that patient, and can use a guide to help him/her determine which implant has the best size and shape for that location, in either a male or a female of that height (assuming that no contrary indications suggest that a different size or shape would be better, for that patient). A pre-manufactured set of implants with specific surface radii for each specific “cam zone” on a femoral runner (as described below) – having the size which has been selected for that specific patient – can be selected by the surgeon, and if a customized size or shape is needed for a specific patient, a custom-fabricated implant can be created (usually, by simply changing certain values that have been inserted into specific data slots, in a computer program that will be used to control a “3D printing machine”, as described below).

It also should be noted that an inventory (or stockpile, supply, or any other suitable term) which contains multiple pre-manufactured implant holding devices as described herein, in a range of different sizes – which can be either: (i) pre-filled, with flexible polymer components, and ready for shipping; or, (ii) stored in an empty form which is ready to receive and hold a flexible polymer insert, if and when an order for an specific type of implant having an exact specified size is received – is an invention in its own right (within the “article of manufacture” category of inventions), and is covered by some of the claims below. That type of inventory – containing hundreds or thousands of such devices, manufactured in large numbers (to achieve the lowest possible cost per unit), and ready for rapid completion and shipping on an “as needed” or “just in time” basis – can be a very valuable asset, for an orthopedic supply company which sells such implants.

Moving on, in this description, any directional terms used herein (such as top, bottom, above, below, etc.) refer to an implant holding device (or complete implant holder) which is in an orientation comparable to a bowl sitting on a table, ready to receive and hold a batch of soup or cereal. In that orientation, the base 20 will be on the bottom, and the open receiving surface of the implant holder (or, the articulating surface of the flexible material 14) will be on the top.

Referring to the Figures, and starting at the upper edge 28, 128, 228 of the sidewall 18, 118, 218, 318 and working downward, the upper edge 28, 128, 228 is not a thin or almost blade-like edge. Instead, it has substantial thickness, since it is designed to accommodate a “setting tool”, which will be positioned directly on top of the upper edge 28, 128, 228 and used (along with a hand-held mallet) to forcibly drive a complete plug implant to the desired depth, during surgical implantation. Also, orthopedic surgeons usually avoid the word “hammer”, as either a noun or a verb, when it comes to their work, since “hammer” has potentially negative meanings and implications, including rough, imprecise, inadequately-controlled, and often abusive acts or behavior. Accordingly, the tools they use for “tapping” (or “impact driving”) operations, intended to drive something into a desired final depth, are called “mallets” rather than hammers.

If the “bone vacancy” (i.e., the hole that has been drilled into a hard bone, to get that bone ready to receive a plug implant) was created with the proper size, it will create a “tight squeeze” when a plug implant is driven into it. Accordingly, the first few taps likely will need to be firm and vigorous, and they will become lighter, and gentler, as an implant approaches its intended final depth. In all tests done to date, on live sheep and in human cadavers, the implants have been driven into the bone, until the upper edge of the implant holding device aligns with the upper surface of the hard bone material; and, the height of the “dome surface” of the flexible material 14 was designed to enable that surface to closely match the height of the undisturbed cartilage which surrounded that insert.

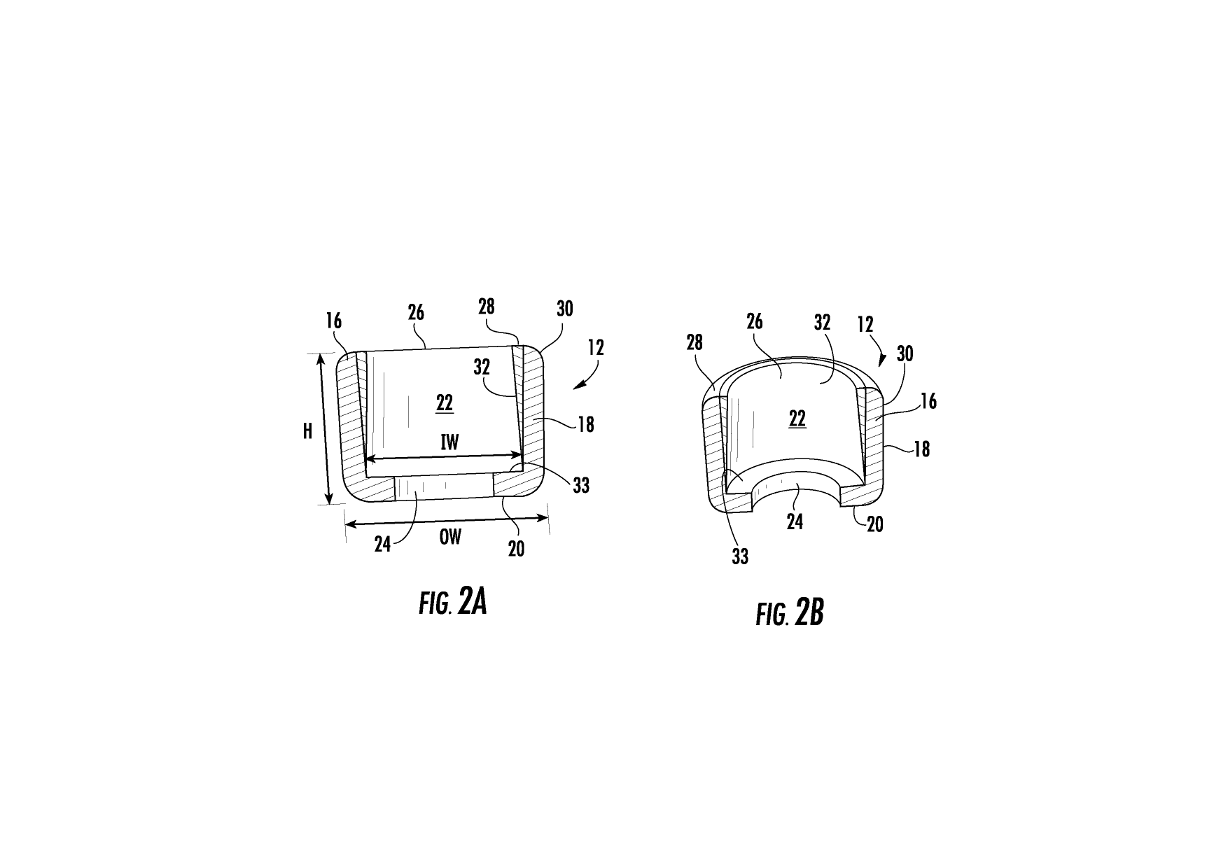

The sidewall 18, 118, 218, 318 includes an outer surface 30. At least a portion of the outer surface 30 will comprise or otherwise be covered by or may be formed into a non-smooth “anchoring surface” 36, also referred to as a “bone attachment layer 36,” that may also be referred to as a bone attachment layer or porous anchoring surface. The outer surface 30 of the sidewall 18, 118, 218, 318 may also have a slightly angled “taper,” as mentioned above. For example, the container body 12, 112, 212 preferably includes a shape in which the outer surface 30 of the sidewall 18 is tapered or otherwise angled or sloped inwardly as the sidewall 18 extends from the top opening 26 to the bottom wall 20. In other words, referring to FIG. 2A, the sidewall 18 is tapered such that the outer width OW varies along the height H of the anchor container body 12, 112, 212, with the outer width OW decreasing in a direction toward the bottom wall 20. This tapered structure of the outer surface 30 allows the anchor 12 to be more easily press-fit into a corresponding cavity formed in a bone. This feature helps to retain the anchor 12 to the bone.

That non-smooth, non-sliding surface (combined with the slightly tapered, wedge-like shape) will help establish a reasonably firm and stable initial gripping response by the hard bone. Indeed, based on both animal and cadaver tests, it is believed that the initial “grip”, created during the surgery and without requiring any recovery time of any sort, will (at least in most cases) be sufficiently strong to enable a patient to stand up and walk away from the operating table, when the surgery has been completed; and, if that can be accomplished, the surgery can be done entirely on an “out-patient” basis, in about two hours or less, in most normal cases. However, regardless of whether the “entirely outpatient” goal can be achieved in any specific patient, the initial level of “grip” will become even stronger over a span of several months, as newly-created hard bony tissue “grows” (i.e., as new bone material is deposited, by cellular secretion activities) into the gaps and vacancies in the porous outer surface 36, acting as a bone attachment layer.

In implants used for tests on sheep, flexible hydrogel materials were loaded into the implant holding devices until the top surface (i.e., the exposed and articulating surface of the complete device) rose to a height of about 1.5 mm, above the upper edge 28, 128, 228, at the highest point in the center of the polymeric insert. In sheep, those implants provided very good results, with no detectable changes in the behavior of the sheep, except for minor alterations in gait, suggesting some tenderness in the leg, for the first few days after the surgery, as should be expected for any type of surgery that involves cutting through the skin and into soft tissues in a “stifle joint” (i.e., the quadruped version of a human knee joint). However, within a few days, any limping or “favoring” of the leg that had been operated on resolved, and disappeared, and after that, the animals stood, walked, and occasionally trotted in an entirely normal manner, which indicated no lingering pain or discomfort of any sort, in the leg which had received the implant.

The porous anchoring surface 36, also referred to as a bone attachment layer 36, on the outside of the sidewall 18, 118, 218, 318 may be formed so as extend only partially along the height of the outer surface of a sidewall, such as shown in FIG. 13, in which the porous anchoring surface 36 extends only partly along the outer surface of the sidewall 318, and a smooth outer surface area 331 is provided between the porous anchoring surface and the upper edge 328. In such aspects, the bone attachment layer 36 may extend about, for example, 2/3 to 3/4 of the height of the sidewall 18, 118, 218, 318. Such a design can have two purposes: (i) it will help reduce the risk that an implant might cause a fracture, splitting, or other failure of the surrounding bone material, when the tapered implant is being forcibly driven into a cylindrical drilled hole; and, (ii) the “shoulder” created by the upper rim of the porous anchoring material can act in a manner comparable to a “barbed” anchoring component. As an aside, orthopedic surgeons know that hard bone material is more similar to a hard wood, than to ceramics or glass, in its response to stress. Hard bone can undergo a significant amount of “give” (i.e., flexing, adapting, etc.), when subjected to various types of stress, rather than suddenly shattering with no warning indications, the way glass will do when stressed to its breaking point. Accordingly, skilled and experienced orthopedic surgeons develop a “sense” or a “feel” for how much stress can be imposed on a bone before it will break, and they learn to stay with that zone of safety, when surgically working with hard bone.

Due to a number of factors, “additive manufacturing” (aka 3D printing) may be used to create all of the plug implants described herein. Among various other advantages, that approach makes it easier and less expensive to modify and “tweak” various aspects of the designs that were being created and tested, to test and evaluate new options, hunches, etc. It is a relatively simple and inexpensive process to simply alter some parameters in a computer program which controls a 3D-printing machine, which will then make a new device having an altered feature or design; and, it is worth noting that the “3D printing” machines that were used could be programmed to create not just one but an entire “queue” of newly modified designs, so that all of them would be ready for filling with a hydrogel, or other polymer, for testing.

However, that does not mean, require, or imply that “additive manufacturing” should or will always be used, or should be used exclusively, to manufacture these types of implants. For example, if and when the manufacturing processes are expanded and adapted to enable the manufacture of large numbers of units, at least some of the components of each implant holding device (such as the barrier layer 38, which can become an initial manufactured component, which can then be modified and added to, in various ways) can be mass-manufacturing by methods which can be used to make hundreds or thousands of copies, per batch, at the lowest possible cost. Subsequently, any additional components or surfaces can then be added to those initial “workpieces”, by methods such as “additive manufacturing,” or in some cases, by using devices such as drills bits, grinding bits, lasers, etc., to remove materials from machined surfaces.

Referring to the Figures and continuing the description starting from the upper edge 28, 128, 228 and moving to the base 20 (while considering the outer surface), such base 20 may have at least one outlet 24 (which also can be called an outlet, a port, an outlet port, a vent, or similar terms), in order to allow any gas or liquid which is in a completed implant holding device, to exit that implant holding device when a flexible insert is inserted into the device (that step is discussed in more detail below). In addition, at least some portion of the outer surface of the base 20 preferably should be covered by a porous material, such as the bone attachment layer, that will encourage bony tissue ingrowth.

Turning now to the inner surface 32 of the sidewall 18, 118, 218, 318, at least some portion of that inner surface 32 must enable a flexible polymer material insert to establish a strong, stable, non-rotating “grip” with the implant holding device which holds it. If a polymer material will be inserted into the implant holding device by injecting a pre-polymerized liquid into the holder, that type of surface can be provided by an irregular surface having protrusions (such as bumps, ridges, fingers, etc.) interspersed with gaps (or troughs, crevices, deficits, vacancies, etc.), in patterns that do not provide any pathways or tolerance for sliding, rotation, or other motion.

However, the complexity and intricacy of the irregularities on that interior surface 32 also must be designed and controlled with an offsetting factor in mind, which is the need to minimize any gas- or fluid-filled “vacancies” that will form between the flexible insert, and the interior surfaces of the implant holding device. The outlet 24 (which passes through the base 20), and various steps used in the filling process (such as creating a strong vacuum inside the implant holding device, before the pre-polymeric liquid is injected into the device), will contribute heavily to that goal, to a point where it can be turned into a negligible factor, especially if a bi-phasic hydrogel is used, which will provide a watery pathway for any pockets of molecular gas to diffuse and escape out of the final implant.

To provide a “better grip” for the flexible polymer inserted into the implant holders, reinforcing or gripping components that can be called struts, ribs, rods, bars, or similar terms, can be provided to traverse at least a portion of the cavity of the implant holding device (with ends of at least some of those struts attached to interior sidewall locations that are spaced apart from each other), and which can provide a small but useful and effective number of “gripping handles” or attachment surface areas for the flexible polymeric material 14 within the cavity in the implant holding device. An array or collection or plurality of such struts can be referred to collectively as a lattice, as further described herein.

Some known approaches, such as perforated plates or a series of walls, might lead to a risk of enabling and possibly contributing to a severe and possibly complete failure of a flexible polymeric insert in such an implant, where the entire polymeric insert might be sheared off of either the top or bottom surface of the perforated plate; and, that risk will become even more serious, if a bi-phasic hydrogel polymer is used, to create the flexible insert in a plug implant.

Accordingly, an approach preferred herein involves placing a reinforcing or “gripping” array of struts 40 (or ribs, bars, or similar terms) inside the cavity 22. Various arrangements of such struts 40 are contemplated. For example, each individual strut 40 can be positioned at a different height (or depth, or elevation), from any other reinforcing strut 40. In another example, certain struts 40 may cross the entire diameter of the cavity (from a first position on the sidewall 18, 118, 218, 318 to an opposite diametrically opposed position across the cavity), and its two ends will be permanently affixed to such diametrically opposed locations, on the inner surface 32. Alternately, certain struts 40 can extend only partially into the cavity.

Some example strut configurations are now discussed. Considering two adjacent struts 40, such struts 40 can be at approximate right angles to each other. If three or four struts are considered, they can look like a six-spoked wheel, when viewed from above, with all of the angles between the “spokes.” Considering three struts, if visualized by comparing them to a round clockface, one strut will pass between the 12 and 6 numbers; one strut will pass between the 2 and 8 numbers, and, the third strut will pass between the 4 and 10 numbers. Also, this approach is analogous to the carpenter’s trick of not hammering all the nails (or driving all the screws) into a piece of wood, along a single line within the “grain” of a beam or plank. Skilled carpenters avoid doing that, since lining up even just two nails with each other, along the grain, can increase the risk that a beam or plank will split apart, along that disrupted and weakened line, in the years or decades that follow, especially if that structure will be subjected to heavy or repeated loadings and stresses during those years or decades. Instead, if two or more nails or screws will be needed, in some particular plank or beam, the better approach is to offset them from each other, in a way that allows the grain of the wood to accommodate each nail or screw as only a single isolated disruption which can be tolerated without damage, rather than as a set of problems which pose a threat of “ganging up” with each other, and which might someday inflict serious damage on the structure being built, if the stresses they must endure cause them to begin acting collectively.

The barrier layer 38 comprising the inner surface 32 of the container body is designed and intended to, for example: (i) reinforce, stabilize, and strengthen the implant holding device; and, (ii) prevent a pre-polymerized liquefied compound from reaching, permeating into, and clogging the porous anchoring layer 36, on the outer surface of either the sidewall 18, 118, 218, 318 or the base 20. Accordingly, it can be designed and regarded as an interior layer, positioned as a barrier in between the patterned “gripping surface” formed by the struts on the inside of the implant holding device, and the anchoring surface 36 on the outside of the implant holding device. It does not need to be especially thick, if made of a strong alloy or polymer, so long as it is impermeable to a liquefied pre-polymer, flexible material such as a hydrogel. Also, it does not need to extend throughout the entire interior of the sidewall 18, 118, 218, 318; instead, it only needs to protect the layer of porous anchoring material 36, to whatever height that outer layer reaches up to, around the sidewall 18, 118, 218, 318.

### THE MANUFACTURE, FILLING AND MOLDING PROCESS

As mentioned above, it is entirely feasible to force-fit a pre-manufactured solid-but-flexible insert component, into a rigid holder; and, that manufacturing method may well be developed and used, some day – if not for all implants, then possibly for only some types of implants, such as implants that are labeled and qualified for use in patients over the age of 70, or who weigh less than some set amount.

A potential optimal method for manufacturing the strongest and most durable types of implants will involve injecting a pre-polymeric liquid into an implant holding device, where the liquid has a sufficient quantity of bonding agents (which also can be called adhesives, adhesive agents, adherents, etc.) to render the mixture able to directly and strongly attach itself to, and adhere to, the internal surfaces of the implant holding device, in a manner comparable to using a tube of liquefied silicone rubber to create a flexible watertight seal between an electrical plug, and an extension cord, to make a waterproof connection that can be left safely outside, where rain or other water can surround it but not penetrate into it.

In addition, it should be noted that all of the fabrication and assembly work which has been done, to date, to create implants for testing in either sheep, or human cadavers, has used an “injection molding” approach, rather than a “force-fitting” approach.

Accordingly, injection molding is the method of manufacture preferred herein.

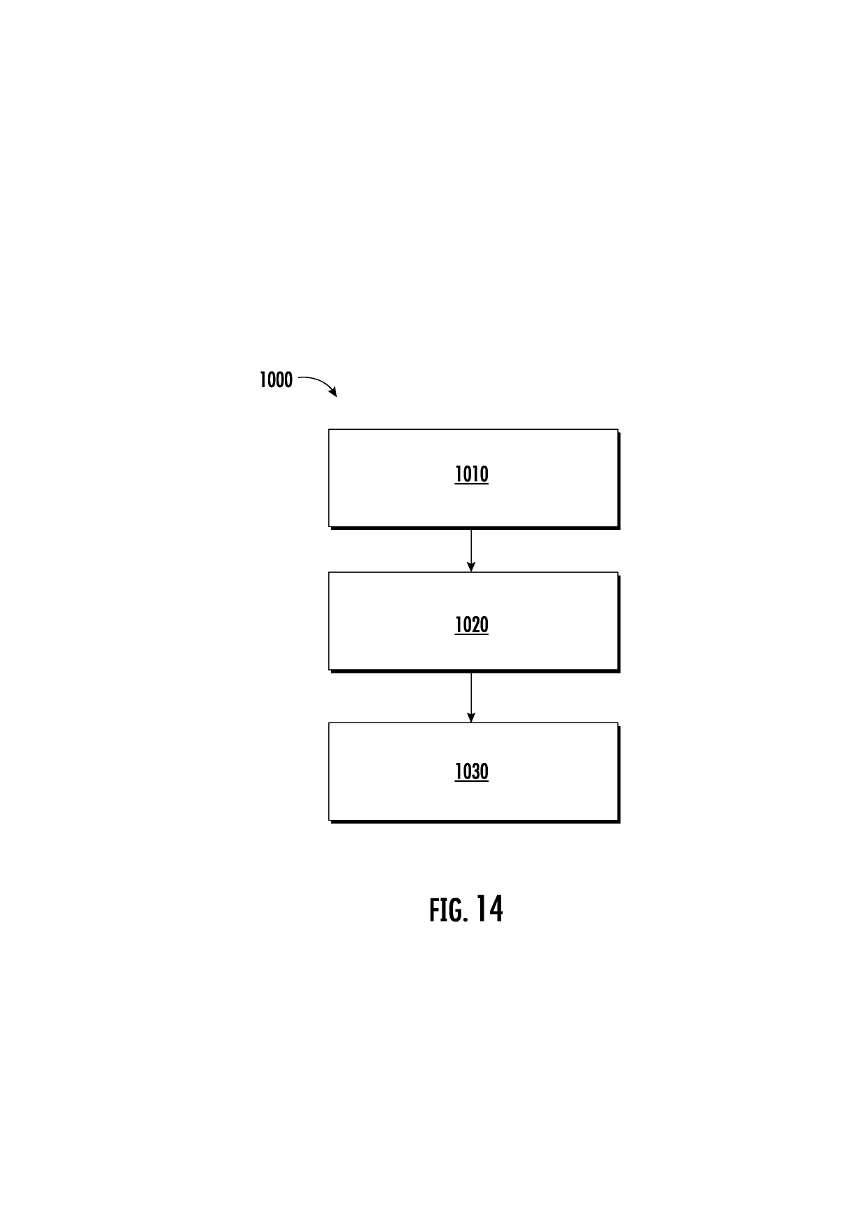

The method 1000, shown schematically in FIG. 14, focuses on creating a small chamber, to hold a single implant holder inside it, with connections to both:
(a) a vacuum pump which could be used to create a strong vacuum inside the chamber, and inside the implant holding device, to minimize any risk of gas bubbles or other “vacancies” being created between the inner walls of the implant holding device, and the polymeric insert which was being created; and,
(b) a supply hose which carried the pre-polymeric liquid to a chosen outlet location.

Accordingly, step 1010 of the method is directed to creating such a chamber.

The chamber is designed to hold the implant holding device with its base near the top of the chamber, and with the “bowl-shaped opening” (and the upper edge) pointing downward. That orientation also allowed the upper edge to be pressed firmly against a machined molding component, which:
(i) matches the diameter of sidewall, so that all liquid into the implant holding device was trapped inside a closed cylinder formed by the combination of the sidewall of the implant holding device, and the molding machine device; and,
(ii) has a slightly bowl-shaped curvature which imparted the desired “dome-like” shape to what would later become the articulating surface of the complete implant, after the pre-polymeric liquid hardened and set into its flexible but solid form.

As described below, various different implants had different “surface radius of curvature” dimensions, in their polymer inserts, and those were created by first creating an assortment of molding components, having diameters to match one of the small, medium, or large implant diameters (which, as mentioned above, were 12, 16, or 20 mm diameters), and also having any of several different “surface radius” dimensions, to provide the assortment of flexible insert shapes that are described below, under the heading, “Size Ranges For Standard-sized Circular Implants.”

When everything has been clamped together, and after a vacuum is created inside the chamber containing an implant holding device, a supply of the pre-polymeric liquid is slowly fed into the bottom of the chamber, where it initially entered and began to fill the “open container” portion of the implant holding device, since the implant holding device was oriented with that large opening near the bottom of the chamber, and with the base (and the vent which passed through the base) at the top of the chamber. The rate of liquid addition, into the implant holding device, should be carefully limited, so that the liquid level inside the implant holding device rises at a rate of only about 1 millimeter every 10 seconds. That gives the liquid enough time to flow into and fill any and all miniature troughs, crevices, depressions, and other openings in the inner surface of the sidewall, to minimize any unwanted vacancies between the polymeric material, and the inner wall of the implant holding device.

Accordingly, step 1020 of the method comprises supplying a pre-polymeric liquid as described.

If desired, a manufacturing chamber (or a tray which holds multiple chambers, to fill multiple devices in each manufacturing batch) can be vibrated, intermittently tapped by a hammering device, rotated intermittently at enough speed to create a substantial or large centrifugal force, or otherwise mechanically bumped and “jarred” or otherwise moved, to help dislodge any bubbles of gas or liquid that might otherwise create vacancies in the polymer. Alternately or additionally, depending on the traits and behavior of a specific pre-polymeric liquid that is being loaded into a implant holding device, the device, chamber, and liquid can be heated to an elevated temperature, to reduce the “viscosity” (which is the liquid version of “thickness” and “resistance to flow”) of the liquid while it is filling the irregular surfaces on the inner walls in the implant holding device.

Accordingly, step 1030 of the method may comprise vibrating or heating the manufacturing chamber.

It also should be noted that the very first quantity of the liquid which enters the two-part molding device (with the upper part being the implant holder, and the lower part being a machined fitting within the molding chamber, which will not become part of the implant) will travel downward and come to rest in the center of a “shallow bowl-shaped” surface, on that molding device. The liquid which comes to rest, touching the “shallow bowl” surface of that molding device, will become the articulating surface of the implant; and, that flexible insert, and its articulating surface, it will have a “surface radius of curvature” which will correspond directly and exactly to the radius of curvature of the shallow bowl surface of that molding component. Therefore, by creating an assortment of several such molding components, with each one having a diameter of either 12, 16, or 20 mm, and with each one having its own radius of curvature, this molding system can create each and all of the standard-sized circular implants described in the next section. Furthermore, this system and the approach it teaches and embodies can be easily adapted, to enable this approach to also create oval-shaped, elliptical-shaped, or custom-shaped plug implants, having any desired shape and dimensions, not just for an assortment of different implant holding devices, but also for an assortment of flexible inserts having any desired articulating surface shapes and sizes that are needed for plug-implant repair of femoral runners in any specific patient (or type of animal).

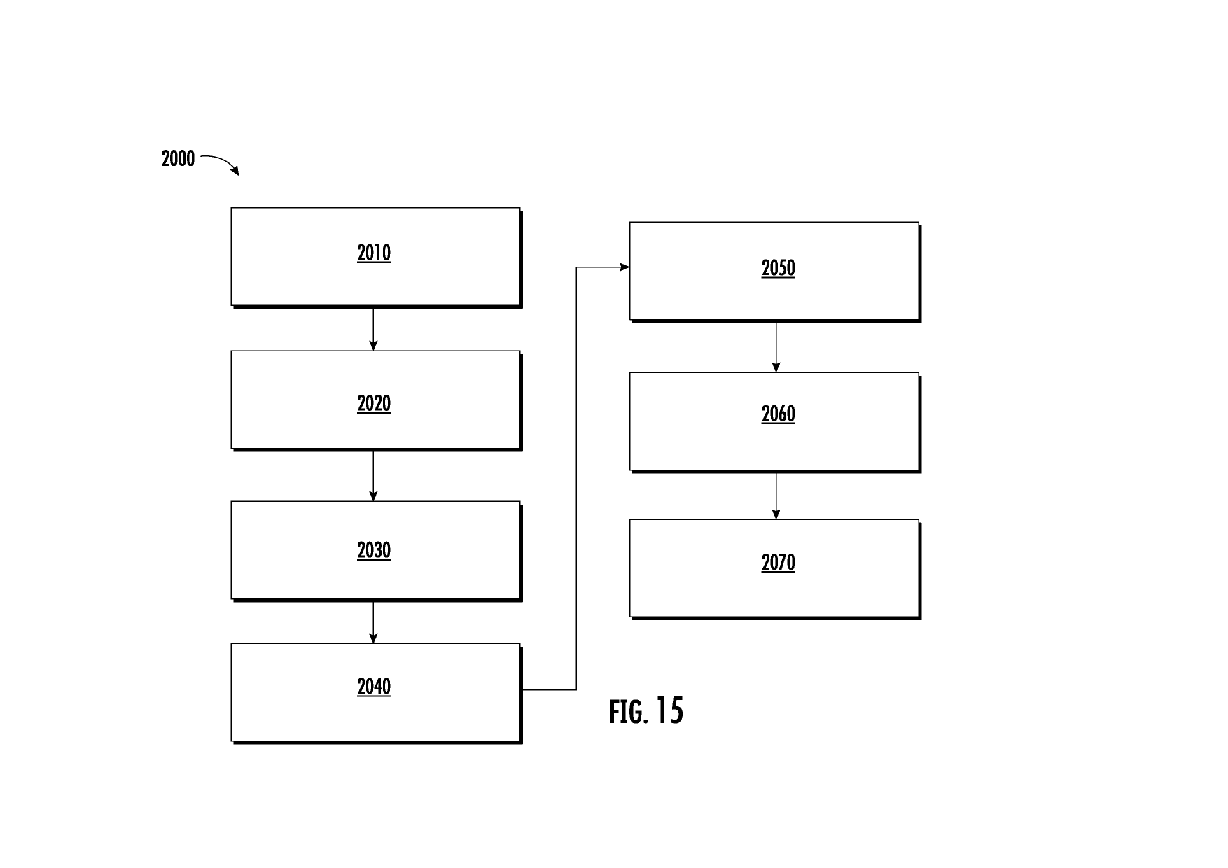

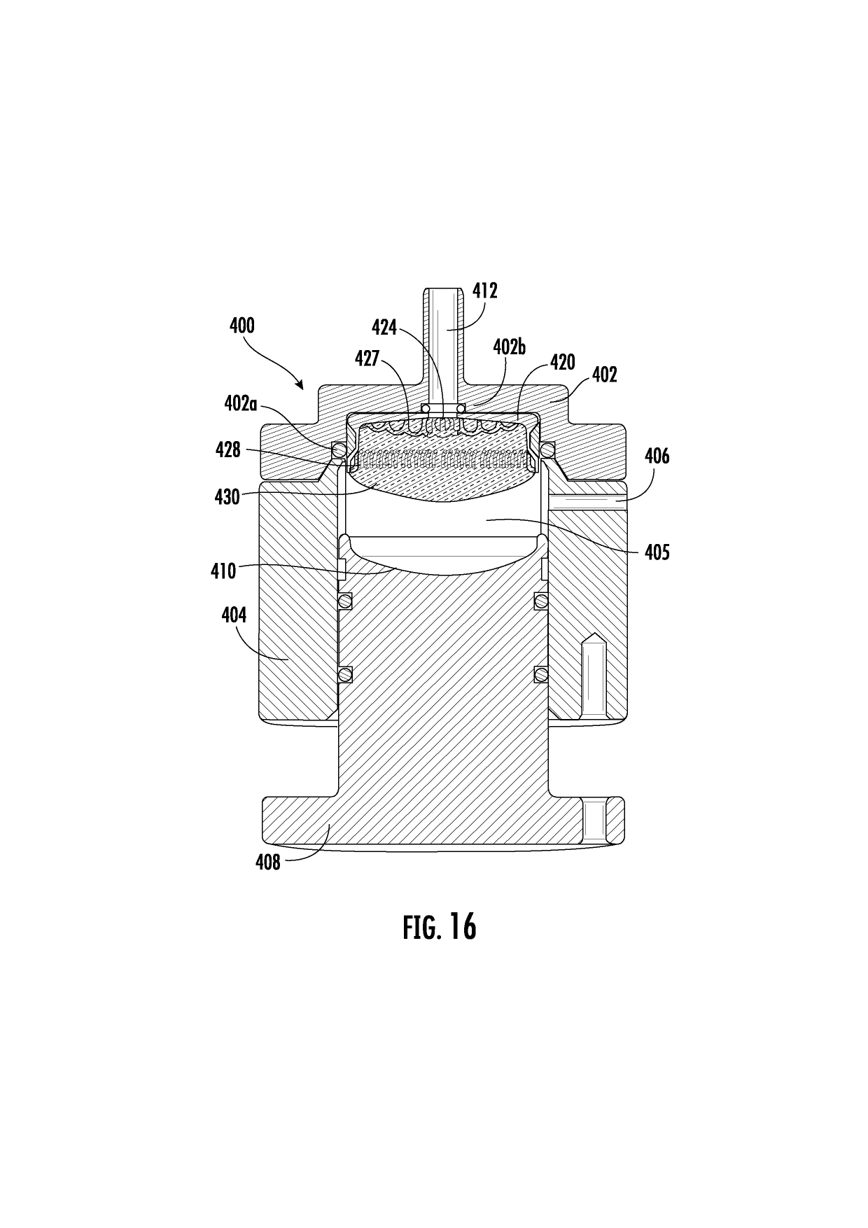

Another example process for forming an implant according to aspects of the present disclosure follows, referring to FIGS. 15 and 16. A method of forming the medical implant 400 includes initially forming the anchor body 420 separately from the articulating component 430 [step 2010]. The anchor body 420 is then placed inside of the first portion 402 such that the bottom wall 422 lays flat on a cup formed by the first portion 402 [step 2020]. The aperture 424 in the bottom wall 422 is aligned with the outlet 412 [step 2030]. The first portion 402 and the second portion 404 are then joined together. As shown in FIG. 13, the first portion 402 and the second portion 404 have complementary interfaces that join each other, and can be joined together [step 2040]. Once the first portion 402 and the second portion 404 are joined together, a flowable material (i.e., hydrogel) is injected via the inlet 406 such that the flowable material fills the space 405 [step 2050]. The flowable material completely surrounds the lattice regions 427, 428 of the anchor body 420. Next, the plunger 408 is driven upwards such that the engagement surface 410 presses into the flowable material to form a top bearing surface of the articulating component [step 2060]. As the plunger 408 is driven upwards, air bubbles in the flowable material are driven upwards and out of the assembly via the outlet 412. The articulating component formed from the flowable material subsequently is partially solidified while retaining elasticity to form a cartilage-like surface [step 2070]. In other words, the articulating component is still a flexible and elastic component that can deform but returns to an undeformed state after a load or stress is applied.

### SIZE RANGES FOR STANDARD-SIZED CIRCULAR IMPLANTS

As mentioned above, one of the main advantages of the novel types of plug implant holders disclosed herein is that they can be mass-manufactured, in advance (to help control manufacturing costs and expenses), in several different sizes.

Those devices will have different diameters, but that was only the start of how they were designed. The Inventor hired a contractor to precisely measure the sizes and shapes of the surfaces on the femoral runners, on 100 different people, and to provide that data in detailed form which could be analyzed on computers. After analyzing and studying the results, and because of the non-circular “cam” shapes of the femoral runners when viewed from the side (also referred to as “along an A-P plane”, where A refers to anterior and P refers to posterior), the Inventor decided that the best outcomes could be achieved if two different design parameters were combined with each other, to create an assortment (or array, grid, or similar terms) of 12 different sizes, which can be shown in a table having 3 rows and 4 columns.

The 3 rows were created by a simple and straightforward decision to create small, medium, and large implants. The diameters that were chosen (based on the Inventor’s experience, from decades as a practicing orthopedic surgeon) were 12, 16, and 20 millimeters.

The four different columns required a more complex analysis, which arose because the Inventor realized that the irregular “cam” shape of a femoral runner, when seen from the side, effectively has five different zones, and each one has a different “surface radius of curvature”; however, one of those five zones rarely becomes damaged, and rarely needs surgical repair, so a set of pre-manufactured standard-sized plug implants would only need special shapes, for the other four zones.

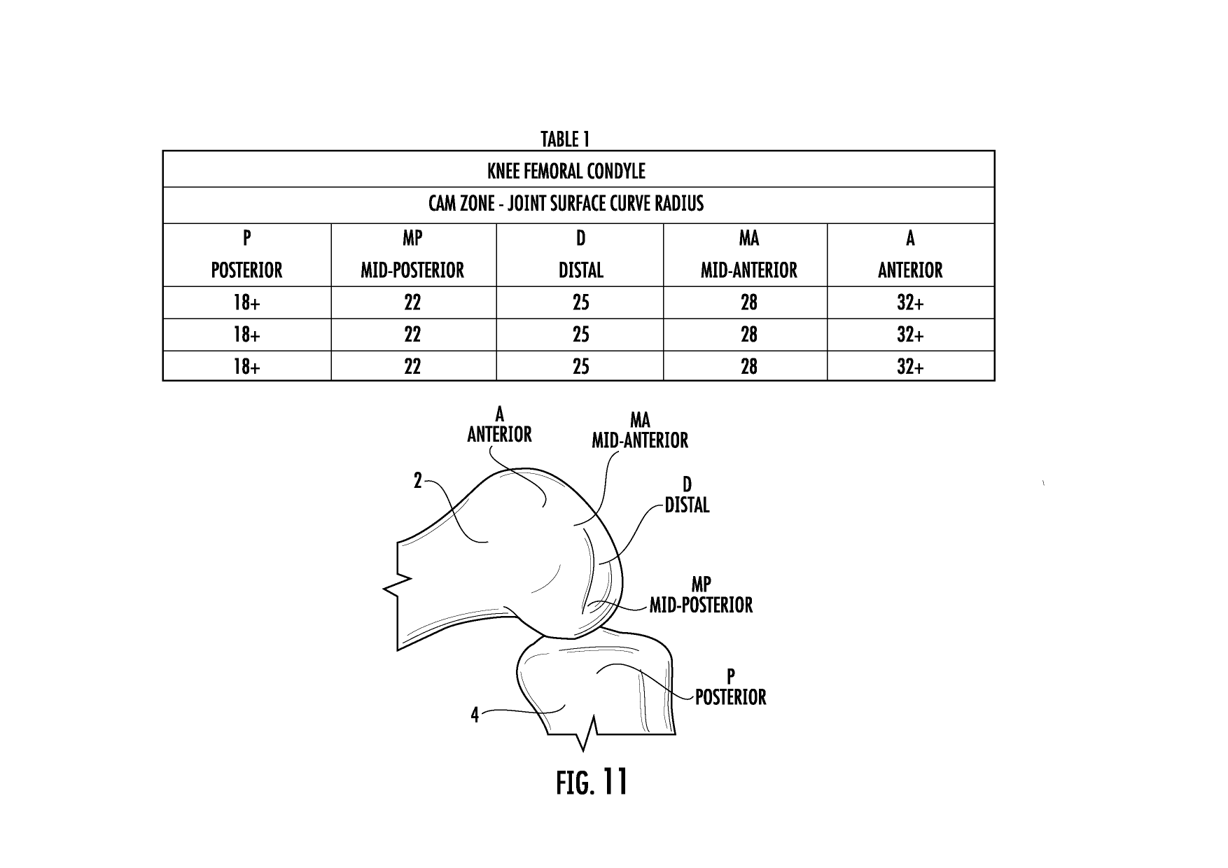

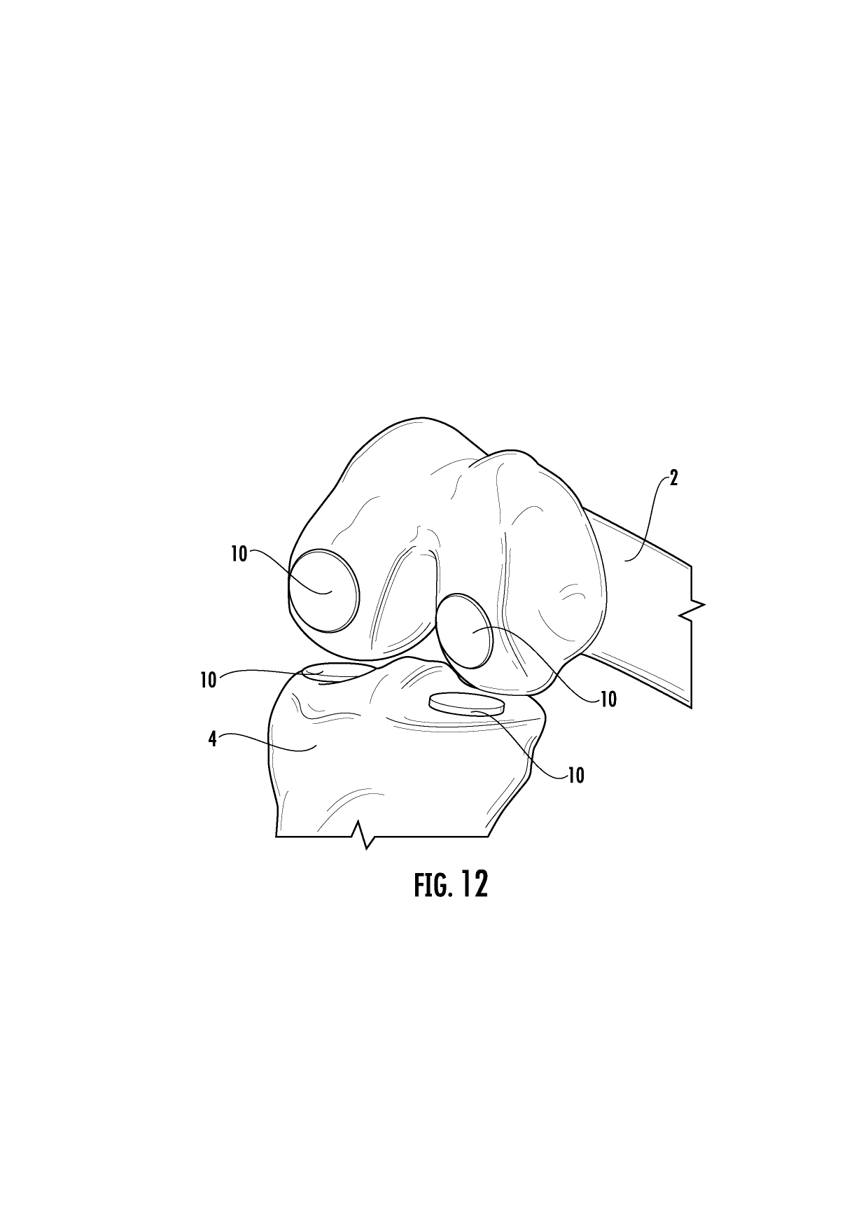

Those four zones (and the names that the Inventor assigned to them) are illustrated in FIG. 11, and can be summarized as follows:
(i) The zone in the “upper front” portion (also called the trochlear facet portion) of a femoral runner is called the anterior zone;
(ii) the zone at the “tip” of the femoral runner (in line with the main axis of the femur bone) is called the distal zone;
(iii) The zone in the back is called the posterior zone; and, because of a number of factors (including a protected location; minimal exposure to traumatic blows; and, the fact that it normally presses and slides against the tibial plateau, only when a person is sitting down, and therefore is not subject to the same levels and durations of weight-bearing loads and stresses that the other zones must endure), it is rarely damaged, and rarely needs repair, so there was no need or effort to create any “standard”-sized plug implants for the posterior zone;
(iv) the zone between the anterior and distal zones was labelled as the mid-anterior zone; and,
(v) the zone between the distal and posterior zones was labelled as the mid-posterior zone.

Each of the four “frequent repair” zones (excluding the posterior zone, as described above), has its own distinct shape; and, that shape cannot be described accurately by referring to a single “radius of curvature”, since it will have one “radius of curvature” when measured along an imaginary “transverse” plane (i.e., an imaginary vertical plane which passes from a person’s right side to his/her left side), and a different “radius of curvature” when measured along an imaginary “anterior-posterior” plane (also called an AP plane), which is a vertical plane passing from front to back.

Nevertheless, the rounded surfaces in each of the four “frequent repair” zones can be approximated, fairly closely, by a single radius of curvature number, where a different number applies in each of the four “frequent repair” zones. Accordingly, the single “best fit” radius of curvature number, for the anterior zone, is 32 millimeters; for the mid-anterior zone, that number is 28 mm; for the distal zone, that number is 25 mm; and, for the mid-posterior zone, it is 22 mm.

The sizes and shapes noted above are not intended to be permanent, as the only types of femoral runner implants that will ever be made. Instead, if the initial set of sizes and shapes listed above is able to prove the utility, durability, functionality, and value of these types of plug implants, any of those initial sizes and shapes can be modified and adapted, easily, into other shapes (including oval or elliptical shapes, if desired) based on actual moldings taken from femoral runners in people having a wide range of different heights and other parameters. As one example, a collection that can include anywhere from 30 to 100 different sizes can be created, which will match, within a few microns in every direction, either the medial runner, or the lateral runner, in either a male, or a female, for anyone within a range of normal heights, divided into “two inch” (or 5 cm) height segments. For example, a first array of sizes can be created for people between 5’4” and 5/6” in height; a second array can be created for people between 5’6” and 5’8” in height; a third set can be created for people between 5’8” and 5/10” in height; a fourth set can be created for people between 5’10” and 6’ in height; and so on, up through people who are 6’10” tall (or even taller, if desired). Similarly, different sets can be created, if a need is seen for doing so, for males versus females, and/or for people having body shapes (or bone thicknesses) that range from “extremely thin” to “extremely thick-set.” Similarly, still more sets can be created, if desired, to handle the minor but measurable differences between the surface shapes of “medial runners” (i.e., on the “inside” of each knee, i.e., the side which is closer to the other knee), versus “lateral runners” (on the outside of each knee). In any such set of molds, the goal will be to use that set of molds to create “plug implants” having surfaces which will be aligned in truly smooth and optimal ways, in all directions, with the undisturbed cartilage which is directly adjacent to any such implant. Stated in alternate terms, the goal is to avoid, at any location around the periphery of a plug implant, the types of slightly mis-aligned, non-smooth junctures and edges that orthopedic surgeons often refer to as “proud edges.”

Some additional specific notes are provided as to certain embodiments as shown and described:

FIGS. 2A-2B illustrate a body, according to aspects of the disclosure. The container body 12 includes a perimeter sidewall 18 (also referred to as the sidewall) and a base 20 connected to the perimeter sidewall 18 on one edge, such as a lower edge, to define a cavity 22 in the interior of the container body 12. The sidewall 18 may preferably be an annular sidewall, such that the anchor has a generally cylindrical shape. The base 20 may further include an aperture 24 communicating with the cavity 22 and a top opening 26 into the cavity 22 defined by an upper edge 28 of the sidewall 18. In this way, the body forms a hollow container section in its interior, formed by the cavity 22, which is configured to receive and retain the flexible material 14, most preferably a hydrogel, therein in a manufacturing or assembly process. The opening or aperture 24 may serve as an aeration hole to allow air to escape when the flexible material is introduced into the top opening 26.

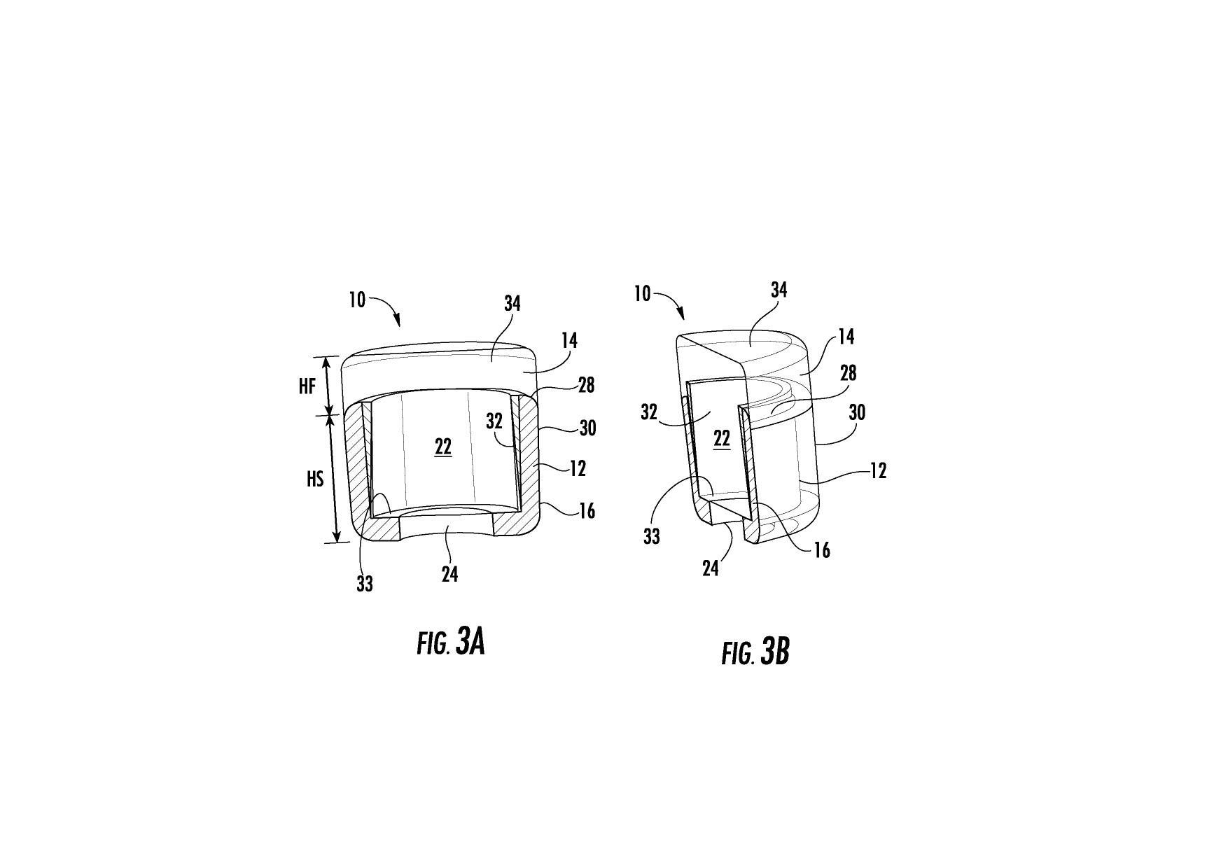

FIGS. 3A-3B illustrate a cross -sectional view of the implant 10, including the anchor 12 with at least a portion of the flexible material 14 extending out of the cavity 22 and above the upper edge 28, preferably covering and in contact with the upper edge 28. The implant 10 defines additional parameters which include a height HS of the cavity 22 (i.e., a height of the inner surface 32) and a height HF which is defined by a distance that the flexible material 14 extends above the upper edge 28 of the anchor container body 12, 112, 212. It can thus be seen that the total height of the flexible material 14 is equal to the sum of the height HS and the height HF. In a preferred embodiment, the height HS is greater than the height HF, as further described.

The flexible material 14 is positioned in the cavity 22 which creates a base for securely attaching to the anchor 12 and for supporting a pad 34 of the flexible material 14 which is the portion of the flexible material which is above the anchor 12 and has the height HF. The pad 34, which may be dome-shaped, is generally the portion of the flexible material above an upper edge 28 of the sidewall 18 which will serve as the replacement cartilage and thus is generally sized to approximate cartilage in the area in which it is being replaced. For example, the height HF of the pad may be approximately 3-4 mm. In an exemplary embodiment, the height HS of the cavity 22 is greater than the height HF of the pad 34, thereby forming a more secure base and attachment member for the pad 34.

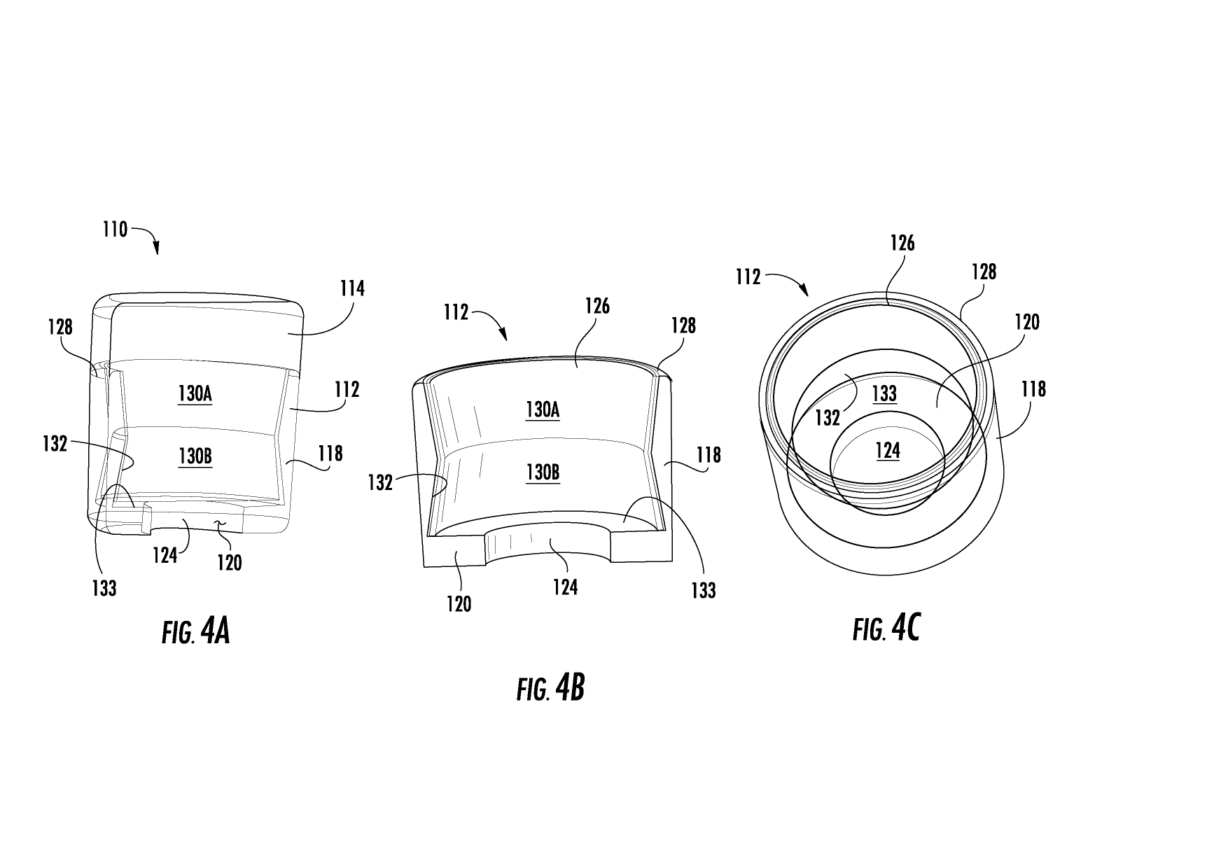

FIGS. 4A-4C illustrate an implant 110 according to another aspect of the present disclosure. The implant 110 is similar to the implant 10, including an anchor 112 and a flexible material 114. The anchor 112 includes a similar structure to that of the anchor 12, including a sidewall 118 and a bottom wall 120 forming a cavity 122, an aperture 124, a top opening 126, and an upper edge 128 (or rim). The sidewall 118 defines an inner surface 132 and the bottom wall 120 defines an inner surface 133. However, an inner surface 132 of the sidewall 118 is different in that it defines a pair of communicating compartments 130A-B which makes up the cavity 122, which has an overall hour-glass-like shape. The upper compartment 130A is flared outward toward the top opening 26, having a wider diameter at its top and a smaller diameter as it extends toward the bottom wall 120. The lower compartment 130B includes a shape which is tapered outwardly as the sidewall 118 extends toward the bottom wall 120. This creates an undercut which is configured to retain the flexible material in the cavity 122. By shifting the tapering to a bottom portion only, a greater taper angle can be achieved without significantly reducing a volume of the cavity 122 (as compared to the sidewall 18 and cavity 22 previously described). In this manner, the portion of the inner surface of the sidewall 118 between the upper and lower compartments can extend a greater distance into the interior of the anchor 112.

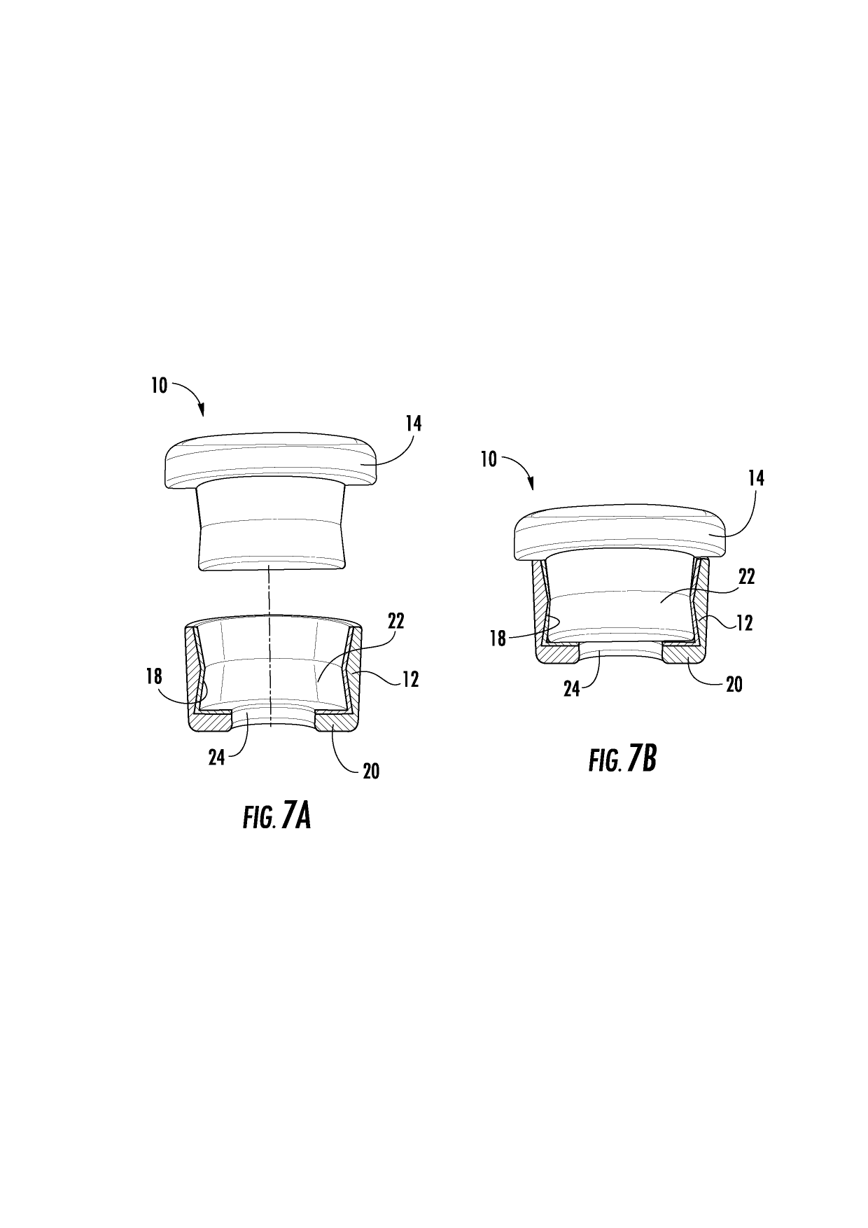

The embodiment of FIGS. 7A-7B provides a modular system in which a container body 12 may be implanted in a bone of an organism to form a site for receiving a flexible material plug or pad which will be used as the replacement cartilage. If the flexible material 14 should become damaged, worn or otherwise no longer be viable, it could be removed from the container body 12 and a new plug or pad installed therein. In this manner, the anchor can remain in place for receiving a new plug or pad of material to replace the cartilage.

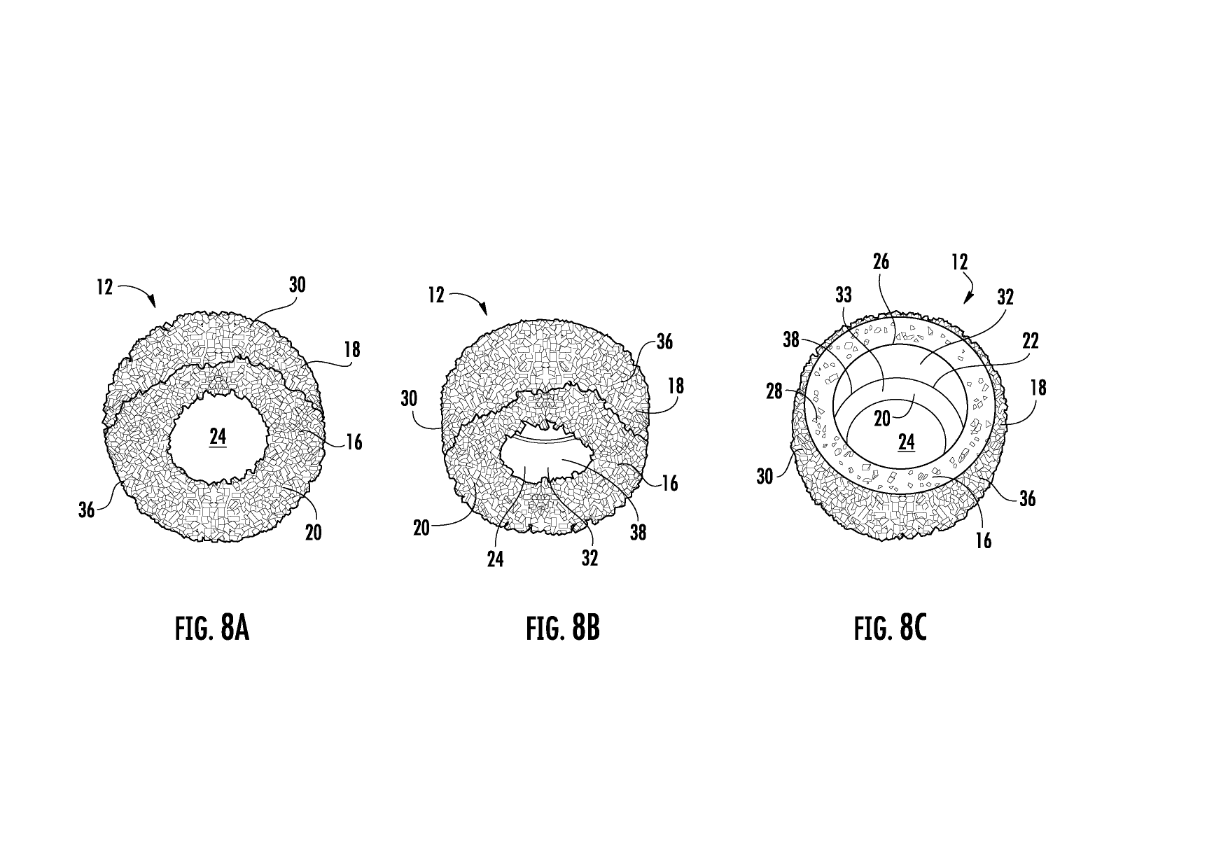

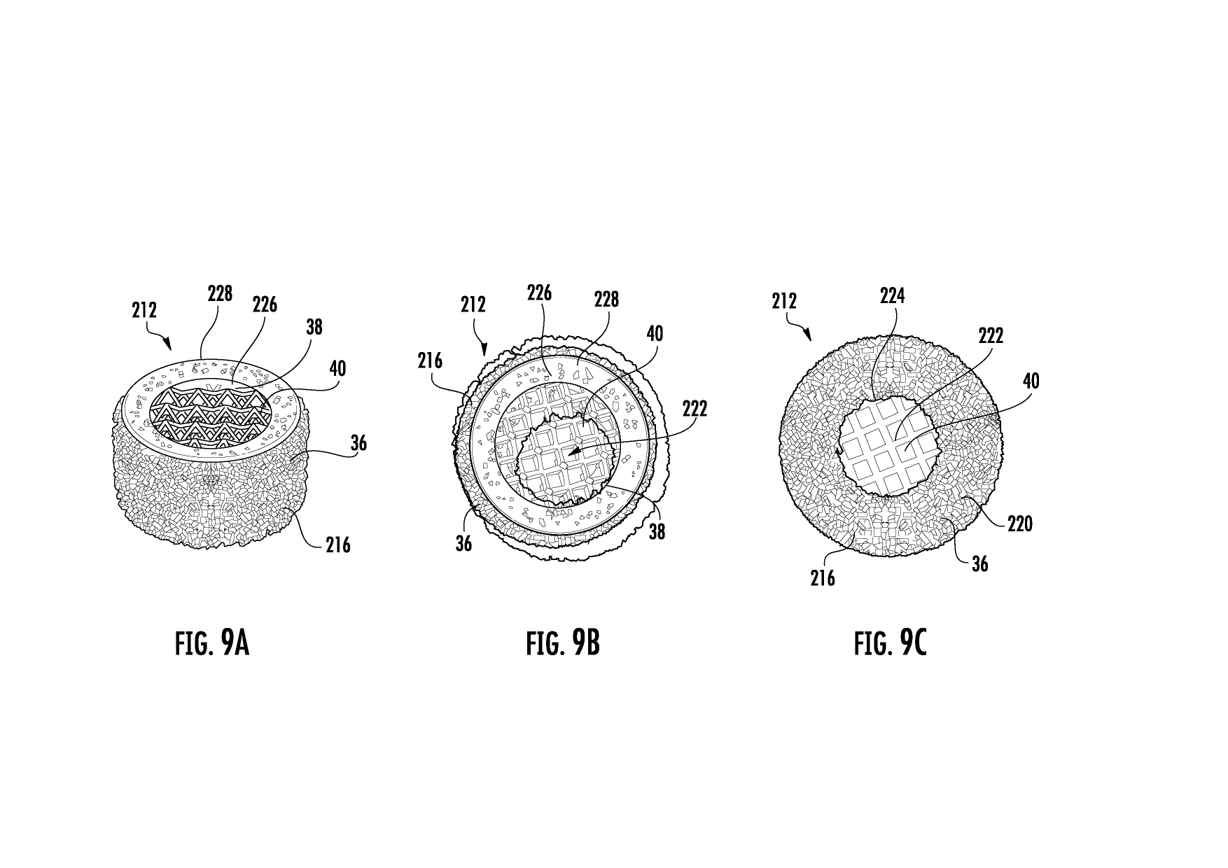

FIGS. 8A-8C and 9A-9C are exemplary illustrations of implant holders such as produced through 3-D printing. FIGS. 8A-8C include the bodies 12, having a sidewall 18, and bottom wall 20. The 3-D printing process produces the anchor body 16 having a bone attachment layer 36 which includes a selected pore size for promoting secure fixation to bone. The 3-D printing process is further capable of forming the container body 12 with a barrier layer 38 formed at the inner surfaces 32, 33 of the sidewall 18 and bottom wall 20 which prevents the flexible material 14 from flowing through to the bone attachment layer 36. In an exemplary embodiment, the barrier layer 38 makes up all or substantially all of the inner surfaces 32, 33 such that the cavity 22 is formed as a receptacle for receiving and retaining the flexible material 14. The upper edge 28 may be formed as a denser or smoother region.

The bone attachment layer 36 is modeled and formed as a trabecular structure which includes interconnected and angled surfaces which form the porous construction of the bone attachment layer 36. The 3-D printing process can overlay a solid barrier layer 38 at the inner surface 32 of the sidewall 18 and/or continue with the same type of trabecular structure only with a smaller pore size which does not allow the flexible material 14 to pass therethrough. Thus, the barrier layer 38 is different in its arrangement, structure or formation of materials as compared to the bone attachment layer 36.

FIGS. 9A-9C include the anchors 212 modeled or produced via 3-D printing. The anchor 212 includes the multi-layer structure as detailed herein, including the bone attachment layer 36, the barrier layer 38, and the array of struts 40. The bone attachment layer 36 and barrier layer 38 are the same or similar to those described in relation to FIGS. 8A-8C. For example, the barrier layer 38 forms the cavity 222 which receives and is impermeable to the flexible material.

The array of struts 40 may be a mesh-like or web-like structure, which creates a porous layer that may be formed through the 3-D printing process. The struts 40 includes a pore or opening size which allows the flexible material to flow through and fill the cavity 222 formed by the sidewall 218. For example, the array of struts 40 includes a pore size of at least 0.4 mm. The array of struts 40 may be positioned anywhere in the cavity 222, including substantially filling the cavity 222.

The implant holders produced via 3-D printing may include the layered structure, which may be made to overlap with each other at the barrier layer. For example, layers (e.g., bone attachment layer 36 and barrier layer 38 on one side and/or the array of struts 40 and barrier layer 38 on the other side) may be formed to overlap by about 0.1-0.2 mm so that the 3-D printing produces solidly adherent layers. The solid barrier layer 38 may be made to be approximately 0.2-2 mm thick, thus producing a layer which adheres the two porous layers to each other and prevents the passage of material therethrough.

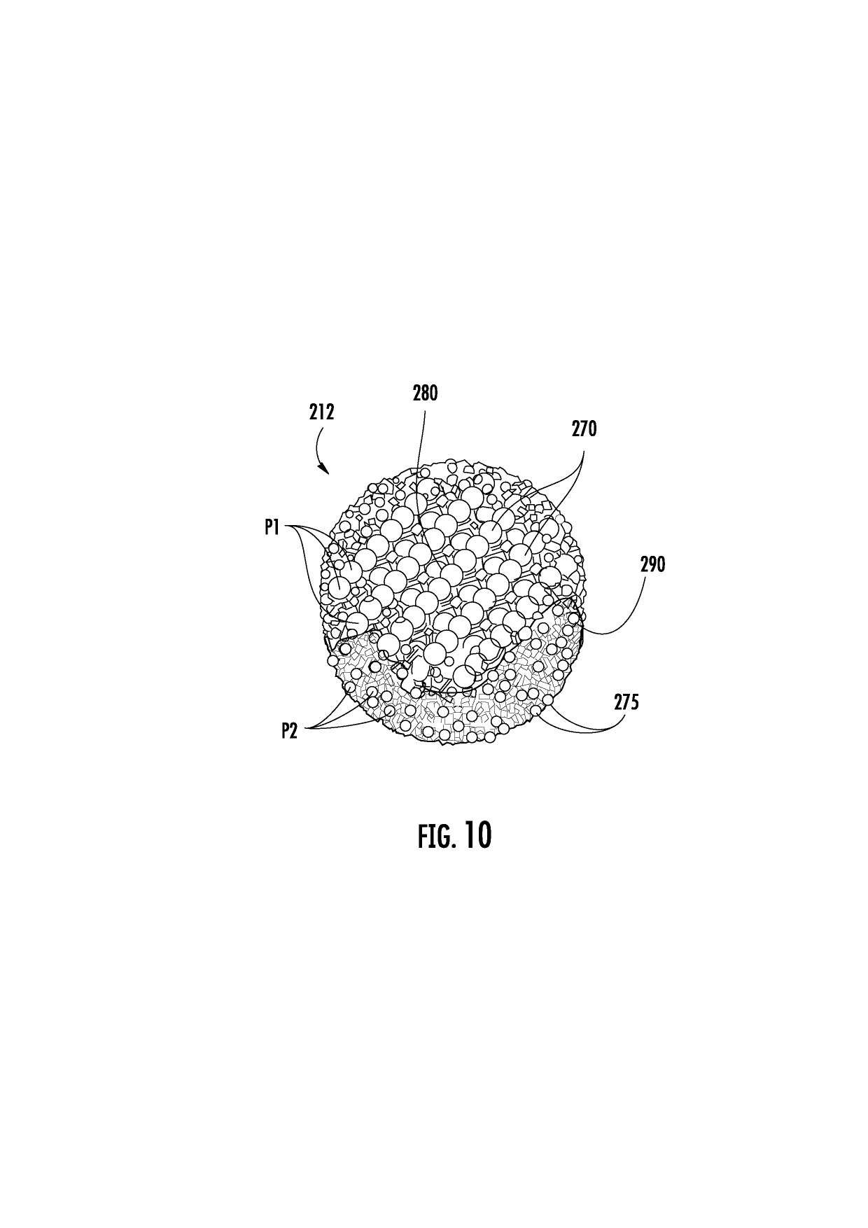

FIG. 10 illustrates the body 212 having larger pore sizes for larger particles 270 in a cavity section 280 than in an outer surface section 290, which includes smaller pores for smaller particles 275. A barrier layer is formed between these sections such that material that can flow through the larger pores in the container section cannot then flow into the smaller pores of the outer surface section. For example, a flexible material cannot flow through the container section 280 into the outer surface section.

Thus, there has been shown and described a new and useful type of implant holding device, which can accept and securely hold any type of cartilage-emulating flexible insert material that any surgeon, company, or researcher wants to test or use, for creating plug-type implants for repairing and replacing a lesion of damaged hyaline cartilage, without requiring an entire segment of cartilage to be removed and replaced. Although the present disclosure has been exemplified for purposes of illustration and description by reference to certain specific embodiments, it will be apparent to those skilled in the art that various modifications, alterations, and equivalents of the illustrated examples are possible. Any such changes which derive directly from the teachings herein, and which do not depart from the spirit and scope of the invention, are deemed to be covered by the present disclosure.

What is claimed is:

1. An implant holding device for a surgical implant, which is designed, sized, and suited to create a plug-type implant which can replace a lesion of damaged hyaline cartilage in a mammalian joint without requiring an entire cartilage segment to be removed and replaced, wherein said implant holding device is designed and suited to:
1a. receive and securely hold a flexible insert component made of a material which will emulate hyaline cartilage and which will provide an articulating surface with a surface shape that will be aligned, in all directions, with all adjacent surfaces of undisturbed hyaline cartilage, to a level which is sufficient to minimize abrasion of an opposing cartilage surface for a period of at least three years after implantation, under normal usage by a recipient who was selected, by an orthopedic surgeon, to receive that type of plug implant to repair damaged hyaline cartilage;
1b. enable said plug-type implant, when complete and holding a flexible insert component, to be forcibly driven into a surgically-prepared vacancy in a bone surface covered by hyaline cartilage, to an exact controllable depth; and,
1c. provide support against compressive, lateral, and shearing forces, for a segment of flexible material which will rise above an upper surface of said implant holding device;
and wherein said implant holding device has a bowl-type shape comprising the following components:
(1) sidewall around its perimeter, wherein:
(i) said sidewall has at least a portion of its outer surface covered by a porous layer of material which will promote bony tissue ingrowth after surgical implantation;
(ii) said sidewall has an inner surface, with at least a portion of said inner surface having a grippable configuration, with a combination of protrusions and deficits which are both: (1) sufficient to enable a flexible material, after it has set and cured, to establish a firm, stable interface between said flexible material and said inner surface of said sidewall; and, (2) large enough to enable a viscous pre-polymeric liquid which has been selected for such use, to enter into (under pressure) and occupy said deficits without creating a quantity of vacancies which would significantly weaken and jeopardize a complete plug implant; and,
(iii) said sidewall also has an internal layer or component which will prevent a viscous liquid from reaching said porous material on said outer surface of said sidewall, while said liquid is being injected under pressure into said implant holding device; and,
(2) a base, having a portion of its outer surface covered by a porous material which will promote bony tissue ingrowth after surgical implantation, and having at least one vent designed to enable a gas or liquid to escape through said vent while a flexible insert component is being added to said implant holding device.

2. The implant holding device of claim 1, which is designed and suited to receive and hold a bi-phasic hydrogel material which contains more than 7% (by weight) of unattached water molecules, wherein said water molecules will be able to move and travel through a matrix of hydrophilic polymer or protein molecules, in response to localized pressure differentials.

3. The implant holding device of claim 1, which has a tapered outer surface shape having a taper angle designed and intended to provide said implant with stronger initial fixation strength and stability as soon as the implant has been impact-driven by an orthopedic surgeon into a prepared cylindrical bone vacancy.

4. The implant holding device of claim 1, wherein said sidewall has an upper edge around its perimeter, at least a portion of which will not be covered by any flexible material after a flexible insert component has been inserted into said implant holding device, wherein said edge has a shape and a thickness that are designed and suited to enable an accommodating tool to be pressed directly against said upper surface, during surgical implantation, in a manner which will enable a surgeon to use impact driving, via the accommodating tool, to drive said implant holding device to an exact desired depth in a cavity that has been surgically prepared in a bone surface beneath a location where damaged hyaline cartilage required surgical repair.

5. The implant holding device of claim 1 which contains at least one internal reinforcing component which extends from one location on said inner surface of said sidewall, to a spaced-apart location on said inner surface of said sidewall, and which is designed to provide additional strength, stability, and durability to a flexible material which is molded into said implant holding device, without creating an elevated risk of creating gas-filled vacancies in a complete implant device.

6. The implant holding device of claim 1, which is designed and suited in all respects for replacing a lesion of damaged hyaline cartilage on a femoral runner, in a knee joint.

7. The implant holding device of claim 1, which is designed and suited in all respects for replacing a lesion of damaged hyaline cartilage on a femoral runner, in a stifle joint on a non-human animal.

8. The implant holding device of claim 1, which is designed and suited in all respects to receive and securely hold a flexible insert component, in a manner which will create a complete implant having a molded articulating surface with a shape which will render said implant suited for replacing a lesion of damaged hyaline cartilage in a human joint other than a knee.

9. The implant holding device of claim 1, which is designed and suited in all respects to receive and securely hold a flexible insert component, in a manner which will create a complete implant having a molded articulating surface with a shape which will render said implant suited for replacing a lesion of damaged hyaline cartilage, in a joint other than a stifle joint, in a non-human animal.

10. The implant holding device of claim 1, wherein said implant holding device is manufactured from a material selected from the group consisting of biocompatible metal alloys, and biocompatible hard polymers.

11. The implant holding device of claim 1, wherein at least one component of said implant holding device is manufactured by an additive manufacturing method.
